(19) United States
(12) Patent Application Publication
(10) Pub. No.: US 2012/0265051 A1
(43) Pub. Date: Oct. 18, 2012

(54) APPARATUS AND METHODS FOR MRI-COMPATIBLE HAPTIC INTERFACE

(75) Inventors: Gregory S. Fischer, Jamaica Plain, MA (US); Hao Su, Worcester, MA (US)

(73) Assignee: WORCESTER POLYTECHNIC INSTITUTE, Worcester, MA (US)

(21) Appl. No.: 13/508,800

(22) PCT Filed: Nov. 9, 2010
(86) PCT No.: PCT/US10/56020

§ 371 Date: Jun. 27, 2012

Related U.S. Application Data
(60) Provisional application No. 61/259,376, filed on Nov. 9, 2009.

(51) Int. Cl. A61B 5/055; G01L 1/24

(52) U.S. Cl. 600/411; 73/800

(57) ABSTRACT

In one embodiment, the system of these teachings includes a master robot/haptic device providing haptic feedback to and receiving position commands from an operator, a robot controller receiving position information and providing force information to the master robot/haptic device, a navigation component receiving images from an MRI scanner, the navigation component providing trajectory planning information to the robot controller, a slave robot driving a needle, the slave robot receiving control information from the robot MRI controller, and a fiberoptic sensor operatively connected to the slave robot; the fiberoptic sensor providing data to the robot controller; the data being utilized by the robot controller to provide force information to the master robot/haptic device. In one instance, the present teachings include a fiberoptic force sensor and an apparatus for integrating the fiberoptic sensor into a teleoperated MRI-compatible surgical system. Methods for use are disclosed.

### BACKGROUND

The present teachings relate generally to the field of haptic feedback, and more particularly, to equipment that is used to measure surgical apparatus insertion force and provide haptic feedback in an magnetic resonance imaging (MRI) guided environment.

MRI-based medical diagnosis and treatment paradigm capitalizes on the novel benefits and capabilities created by the combination of high sensitivity for detecting tumors, high spatial resolution and high-fidelity soft tissue contrast. This makes it an ideal modality for guiding and monitoring medical procedures including but not limited to needle biopsy and low-dose-rate permanent brachytherapy seed placement. MRI compatibility necessitates that both the device should not disturb the scanner function and should not create image artifacts, and that the scanner should not disturb the device functionality. Generally, the development of sensors and actuators for applications in MR environments requires careful consideration of safety and electromagnetic compatibility constraints.

A number of MRI-guided surgical procedures may be assisted through mechatronic devices that present more amiable solution than traditional manual operations due to the constraints on patient access imposed by the scanner bore. However, the lack of tactile feedback to the user limits the adoption of robotic assistants.

Often the interventional aspects of MRI-guided needle placement procedures are performed with the patient outside the scanner bore due to the space constraint. Removing the patient from the scanner during the interventional procedure is required for most of the previously developed robotic systems. There is a need for needle motion actuation and haptic feedback in order to greatly improve the targeting accuracy by enabling real-time visualization feedback and force feedback. It may also significantly reduce the number of failed insertion attempts and procedure duration.

During needle interventional procedures, traditional manual insertion provides tactile feedback during the insertion phase. However, the ergonomics of manual insertion are very difficult in the confines of an MRI scanner bore. The limited space in closed-bore high-field MRI scanners requires a physical separation between the surgeon and the imaged region of the patient. In addition to the ergonomic consideration, by allowing the surgeon to operate outside the ore they would have access to seeing MRI images, navigation software displays, and other surgical guidance information during needle placement. For example, in a biopsy case, real-time MRI images would be shown to the surgeon and augmented with guidance information to help assist appropriate positioning. In brachytherapy radioactive seed placement, information including real time dosimetry would be made available. Force feedback would help to train inexperienced surgeon to learn important surgical procedures and significantly increase the in-situ performance.

Many variants of force sensors are possible, based on different sensing principles and application scenarios. A hydrostatic water pressure transducer was developed to infer grip force and a 6-axis optical force/torque sensor based on differential light intensity was used for brain function analysis. A large number of fibers are necessary in this design and its nonlinearity and hysteresis are conspicuously undesirable. A novel optical fiber Bragg grating sensor was developed and it is MRI-compatible with higher accuracy than what is typically necessary and has high cost support electronics. None of the aforementioned force sensors (except the high-cost fiber Bragg sensor) satisfy the stringent requirement for needle placement in MR environment. There is a need for a cost-effective MRI-compatible force sensor.

### SUMMARY

The needs set forth herein as well as further and other needs and advantages are addressed by the present embodiments, which illustrate solutions and advantages described below.

In one embodiment, the system of these teachings includes a master robot/haptic device providing haptic feedback to and receiving position commands from an operator, a robot controller receiving position information and providing force information to the master robot/haptic device, a navigation component receiving images from an MRI scanner, the navigation component providing trajectory planning information to the robot controller, a slave robot driving a needle, the slave robot receiving control information from the robot controller, and a fiberoptic sensor operatively connected to the slave robot; the fiberoptic sensor providing data to the robot controller; the data being utilized by the robot controller to provide force information to the master robot/haptic device.

In one instance, the present teachings include a fiberoptic force sensor and an apparatus for integrating the fiberoptic sensor into a teleoperated MRI-compatible surgical system. One embodiment of the sensor has hybrid (one axis force and two axis torque) sensing capability designed for interventional needle based procedures. The apparatus of the present teachings includes, but is not limited to force monitoring and haptic feedback under MRI-guided interventional needle procedures, which significantly improves needle insertion accuracy and enhance operation safety.

The system of the present embodiment includes, but is not limited to, system arrangement in MRI environment, an optic force sensor, a modular haptic needle grip, teleoperation control algorithm, a robotic needle guide and force feedback master device.

Other embodiments of the system and method are described in detail below and are also part of the present teachings.

For a better understanding of the present embodiments, together with other and further aspects thereof, reference is made to the accompanying drawings and detailed description and its scope will be pointed out in the appended claims.

### BRIEF DESCRIPTION OF THE DRAWINGS

FIG. 1 is a schematic illustration depicting one embodiment of the system architecture with the slave robot and controller inside the MRI scanner room and the master device and operator are outside the scanner room;

FIG. 2 is a schematic illustration depicting one embodiment of the system architecture with the entire haptic system operating within the MRI scanner room;

FIG. 3 is a schematic illustration depicting one embodiment of the system architecture with the slave robot, haptic master, and robot controller operating within the MRI scanner room and the navigation software interface outside the scanner room;

FIG. 4a and FIG. 4b are schematic illustrations depicting one embodiment of the master-slave teleoperation framework;

FIG. 5 is a pictorial depicting one embodiment of a haptic needle grip;

FIG. 6 is pictorial depicting one embodiment of a 3-DOF force/torque sensor structure;

FIG. 7a is pictorial depicting one embodiment of the light reflection by spherical mirror from central emitter fiber, and FIG. 7b is pictorial depicting the simulated received light intensity change with different mirror translation/rotation;

FIGS. 8a and 8b are pictorials depicting one embodiment of an interferometry force sensor interface;

FIG. 9a is a pictorial representation of forces acting on a needle, and FIG. 9b depicts a typical in-vivo prostate needle insertion force profile;

FIG. 10a and FIG. 10b are pictorials depicting a configuration of a needle insertion robot in an MRI scanner with a patient;

FIG. 11 is a pictorial representing one embodiment of an MRI-compatible needle placement robot;

FIG. 12a is a pictorial representing one embodiment of an MRI-compatible needle insertion module, and FIG. 12b is a pictorial representing one embodiment of a needle driver; FIG. 12c is a block diagram representation of one embodiment of a needle placement robot of these teachings;

FIG. 13a is a pictorial representing one embodiment of a needle rotation unit, and FIG. 13b is a pictorial representing one embodiment of a needle clamp;

FIG. 14a is a pictorial representing one embodiment of a needle driver with lateral needle force sensing, FIG. 14b is a pictorial representation of an alternate embodiment of a lateral needle force sensor, and FIG. 14c is a pictorial representation of a needle driver base with axial force sensing;

FIG. 15a is a pictorial representation of a 1-DOF haptic master device, and FIG. 15b is a pictorial representation of a multi-DOF haptic master device; FIG. 15c is a block diagram representation of an embodiment of a master device of these teachings;

FIG. 16 is a block diagram representing an embodiment of the method of these teachings;

FIG. 17 is a flow chart representing one embodiment of the work phases during a needle placement procedure;

FIG. 18 is a flow chart representing one embodiment of the work phases during a needle placement procedure.

### DETAILED DESCRIPTION

The present teachings are described more fully hereinafter with reference to the accompanying drawings, in which the present embodiments are shown. The following description is presented for illustrative purposes only and the present teachings should not be limited to these embodiments.

In this document, needle is defined as long-shaft surgical instrumentation that provides axial translational and rotation motions and interact with soft tissues, including but not limited to medical needles, electrodes, ablation probes, tissue sensors, tubes, guide sleeves, and canulae.

A “robot,” as used herein, is an electro-mechanical or mechatronie device which is guided by computer or electronic programming.

A “master-slave” system, as used herein, refers to a system in which the operator manipulates a “master” device and the operation of the “master” device is translated into instructions provided to the “slave” robot, the instructions resulting in the “slave” robot performing a task.

“Compatible with the MRT environment” or “MRI-compatible,” as used herein, refers to devices that substantially preserve the image quality of the scanner and whose operation is substantially not affected by the high field MRT environment.

“Light,” as used herein, refers to electromagnetic radiation without limitation to visible wavelength.

A “force sensor,” as used herein, refers to a sensor that measures force and/or torque along or about one or more axes.

One specific application of the system and apparatus is a semi-automated needle guide for MRI-guided prostate brachytherapy and biopsy with haptic feedback. These teachings can be generically applied to other procedures including needle-based percutaneous procedures under other medical imagers, including but not limited to ultrasound, computed tomography (CT), fluoroscopy, X-ray.

In one embodiment, to overcome the loss of tactile feedback in a robot-assisted insertion, needle tip force information, these teachings present a teleoperated force feedback system with fiberoptic force/torque sensor, to be integrated with a robotic needle guide for MRI-guided prostate needle placement. A navigation and control framework integrated with an MRI-compatible fiberoptic force sensor embodiment can be leveraged to close the sensing and control loop in a teleoperation manner.

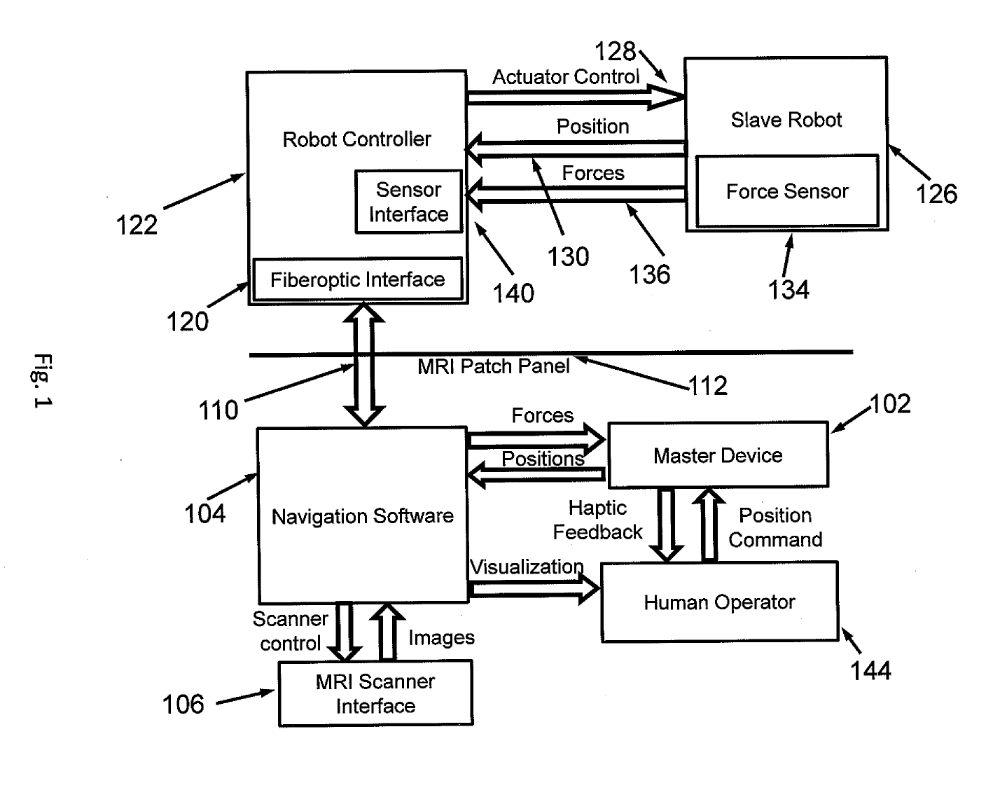

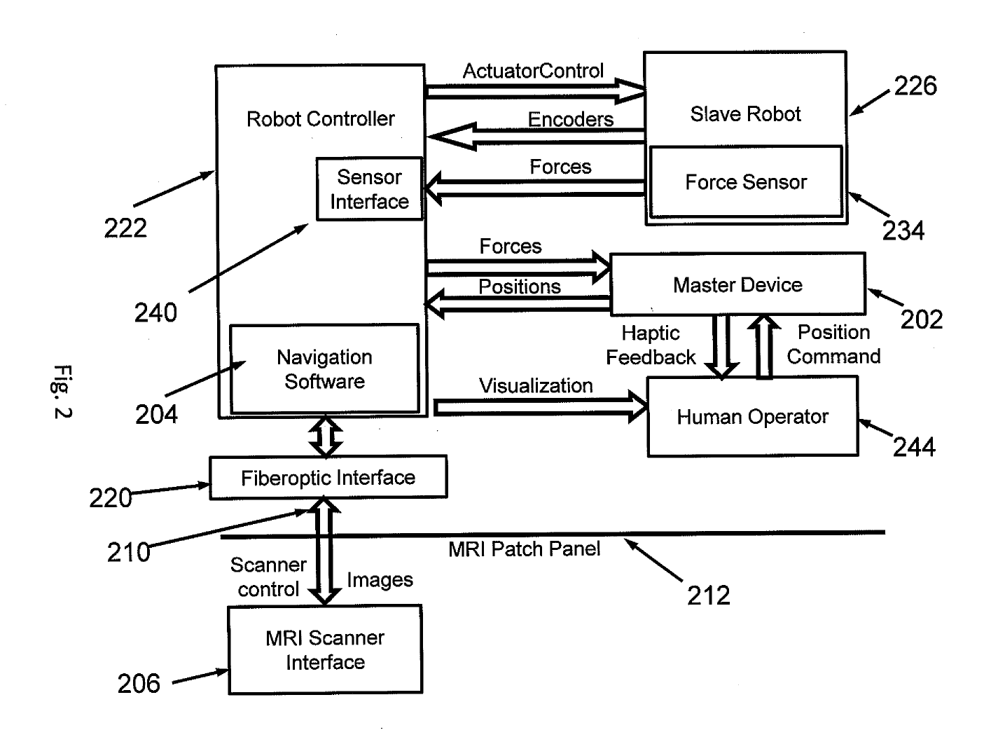

In one system architecture to utilize a haptic interface in MM as shown in FIG. 1, a haptic master device 102 and a navigation software interface 104 and a scanner interface 106 reside in the console room. Navigation software 104 runs on a computer and is communicatively coupled through fiberoptic connection 110 through MRI patch panel 112 to fiber media converter or interface 120 inside of robot controller 122. Robot controller 122 is MRI-compatible and resides inside the MRI scanner room. In one embodiment, it contains a communication interface, power regulation, a computer, sensor interfaces, and actuator interfaces. The slave robot 126 operates within the MRI scanner bore and receives actuator control or power 128 and feeds back position information 130. Actuator control signal 128 may include but not be limited to piezoelectric actuation, pneumatic actuation, and hydraulic actuation. Position sensing 130 may include but not be limited to optical encoders, fiberoptic sensors, and potentiometers. Alternatively, position sensing may be image-based and determined from images. Slave robot 126 incorporates one or more force sensors 112 for measuring tissue interaction forces. The force sensor may measure one or more of axial insertion force and lateral forces. In one embodiment, the force sensor is a fiberoptic force sensor. In a further embodiment, the slave robot 126 includes a needle insertion module capable of sensing 1-DOF axial needle insertion force and 2-DOF lateral forces on the needle body. Force sensor is coupled by connection 136 to sensor interface 140. In one embodiment, connection 136 includes is a fiberoptic cable. The sensor interface 140 may reside inside robot controller 122, elsewhere in the MRI scanner room, or as a standalone interface in the console room. Sensor interface 140 may couple directly to navigation software interface 104. In one embodiment, optic force sensor interface 140 is incorporated into the robot controller and the needle interaction forces measured by a fiberoptic force sensor 134 are transmitted back to the navigation software console 104 along with the robot position. In one configuration, the haptic feedback device is integrated into the navigation software framework (for example, but not limited to, the software as described in Gering et al., An integrated visualization system for surgical planning and guidance using image fusion and an open MR, J Magn Reson Imaging, 2001 June; 13(6):967-75, which is incorporated by reference herein in its entirety for all purposes; Pieper, S.; Halle, M.; Kikinis, R.; “3D Slicer,” Biomedical Imaging: Nano to Macro, 2004. IEEE International Symposium on, vol., no., pp. 632-635 Vol. 1, 15-18 Apr. 2004, which is Incorporated by reference herein in its entirety for all purposes; Tokuda Fischer G S, DiMaio S P, Gobbi D G, Csoma C, Mewes P W, Fichtinger G, Tempany C M, Hata N, Integrated Navigation and Control Software System for MRI-guided Robotic Prostate Interventions, Computerized Medical Imaging and Graphics, August 2009; which is incorporated by reference herein in its entirety for all purposes) to provide forces to the operator and control back to the robot. In an alternative embodiment, the fiberoptic sensor 134 may communicate with a controller outside the scanner room or the force sensor interface may be a stand-alone device. In a further embodiment, the robot controller 122 is outside the MRI scanner room and signal 128, 130, and 136 are passed through the patch panel 112 or other location to the MRI console room. Further, robot controller 122 and navigation software 104 may reside on the same physical computer with no external interconnect.

In one embodiment of the system architecture shown in FIG. 1, a commercially available haptic device 102 (such as, for example, a device as disclosed in U.S. Pat. No. 7,103,499, which is incorporated by reference herein in its entirety for all purposes, or a Novint Falcon haptic device; see, for example, Steven Martin, Nick Hillier, Characterisation of the Novint Falcon Haptic Device for Application as a Robot Manipulator, Australasian Conference on Robotics and Automation (ACRA), Dec. 2-4, 2009, Sydney, Australia, which is Incorporated by reference herein in its entirety may be used as the master robot. In one configuration, the master has 6 Cartesian DOF and can be used to position and orient the needle. Other numbers of DOF of sensing and feedback may be used. A human operator position obtained from the haptic interface is used for trajectory generation and control of the motion of the slave robot 126. In one embodiment, the slave robot is a 6-DOF robotic assistant for intraprostatic needle placement inside closed high-field MRI scanners. Force feedback enables an actuated needle driver and biopsy firing mechanism and needle rotation. Contact forces between needle and tissue may be measured by the fiberoptic force sensor 134 and fed to the haptic device. The sensor may measure insertion forces along the needle axis, lateral forces, torques about the needle axis, and/or lateral torques. One embodiment of sensor 134 measures insertion force and lateral force/torques to help guide the insertion procedure.

In one embodiment, the master 102 device resides outside the MRI scanner room. In one configuration, it resides in the adjacent console room. In an alternate embodiment, one or more of the haptic master 102 and navigation software interface 104 are in a remote location. Master 102 receives force control signals corresponding to the sensed forces from sensor 134. The forces may be directly fed to the master or augmented before being fed back to operator 144 who interacts directly with master 102.

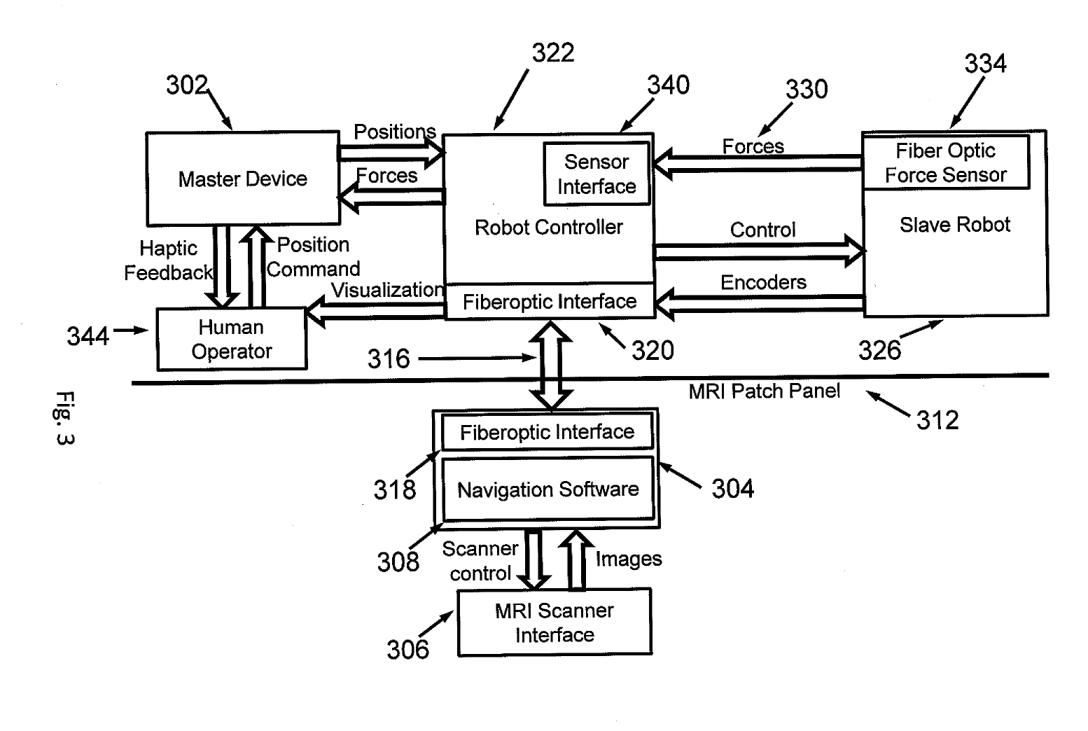

In one embodiment, both an MRI-compatible master device 202 and an MRI-compatible slave robot 226 are located inside the MRI scanner room as shown in FIG. 2. In one configuration of this embodiment, a robot controller 222 resides inside the MRI scanner room and is connected to both the slave robot and the master device 202. The robot controller powers the slave robot actuators, reads the position sensors, and measures forces. In one configuration, forces are measured by fiberoptic force sensor or sensors 234 though sensor interface 240. The robot controller 222 includes the sensor and actuator interfaces and joint level control software. In one embodiment, the robot controller also includes a computer. In one configuration, the navigation software 204 resides on the robot controller which is communicatively coupled by converter interface 220 and connection 220 through patch panel 212 to the MRI scanner interface 206. In one configuration, the robot controller 222 communicates with an MRI scanner interface 206 via fiberoptic interface 220 using fiberoptic cables 206. In a further embodiment, the navigation software 204, which may reside on the robot controller 222 or on another computer, both retrieves MR images from MRI scanner interface 206 and also controls the scanner. Scanner control can include, but is not limited to, scan parameters, slice location and slice orientation. Scanner control may be used to actively track a needle or target during a needle insertion procedure so that both visual and haptic feedback may be provided to the clinician.

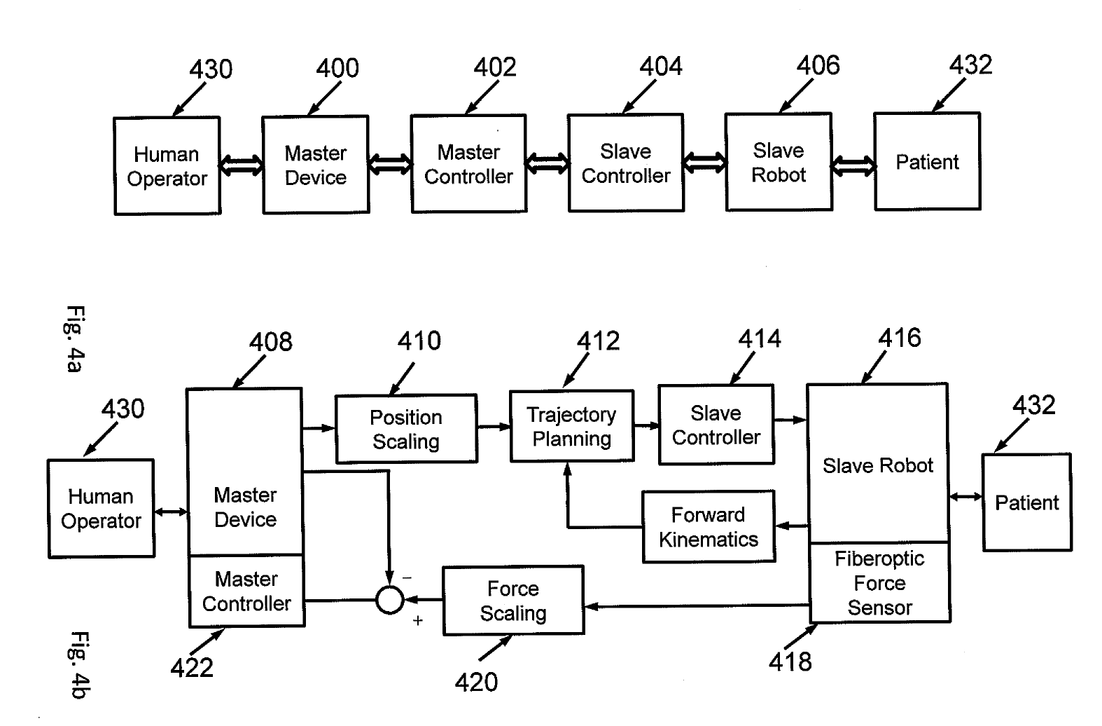

In a further embodiment, shown in FIG. 3, the robot controller 322, the slave robot 334, the master device 302, and the operator 344 reside inside the MRI scanner room, and the navigation software computer 304 resides outside the scanner room. Master device is MRI-compatible and operates within the MRI scanner room. Haptic master 302 interacts with user 344 to receive commands and provide tactile feedback. Haptic master 302 applies forces using MRI-compatible actuators to the operator 344 which are measured by an optical force sensor. Position of the master device 302 is reflected in the slave robot 326 that follows and measures interaction forces with the tissue with optical sensor 334. The forces sensed by the slave are fed back to the master as a bilateral teleoperator through the robot controller 322. Visualization may be provided to the operator from the robot controller 322, from an external display coupled to the navigation software computer 304, or another source. Robot controller 322 contains actuator interfaces, sensor interfaces, a computational unit, and a communication unit. In one configuration, the communication unit is a fiberoptic network interface 320 that communicates via fiberoptic cables 316 though MRI patch panel or wave guide 312. In the MRI console room resides a control computer or other device 304 that contains a communication interface 318 that communicates with robot controller 322. In one embodiment, the control computer 304 in the MRI console room runs navigation and control software 308. Visualization may be provided in the console room and may also be on an MRI-compatible display inside the MRI scanner room with the patient and operator. Both slave robot 326 and master device 302 are MRI-compatible and the slave robot 326 is equipped with fiberoptic sensor or sensors 334 which communicates with robot controller 322 though sensor interface 340 that can be standalone or be integrated with robot controller 322. The robot controller is communicatively coupled to the MRI scanner computer, imaging server, navigation software workstation, or other interface via a fiberoptic network interface 320 by fiberoptic cables 316 that passes though MM patch panel or other access location 312.

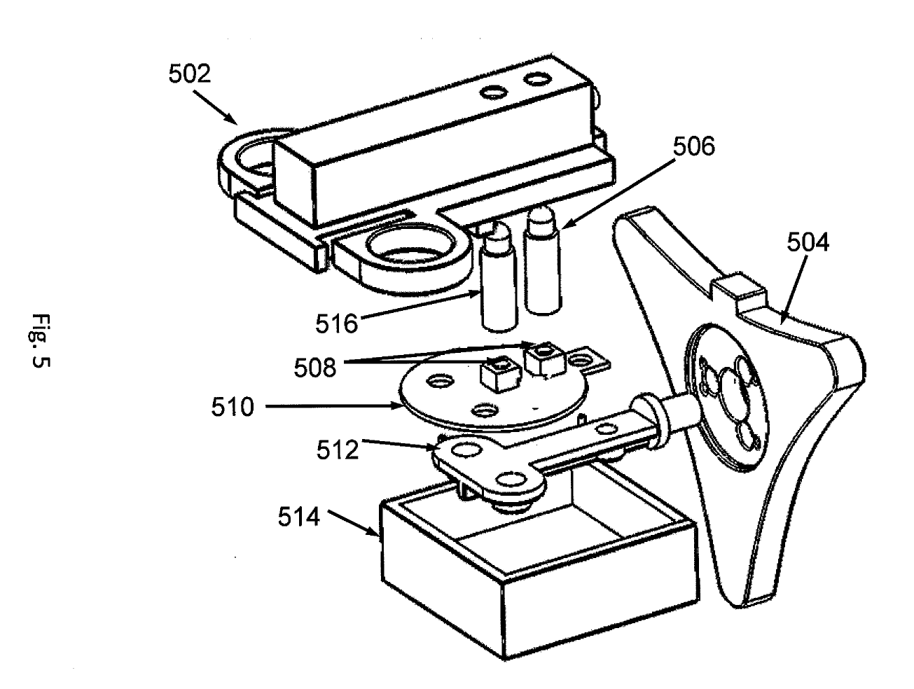

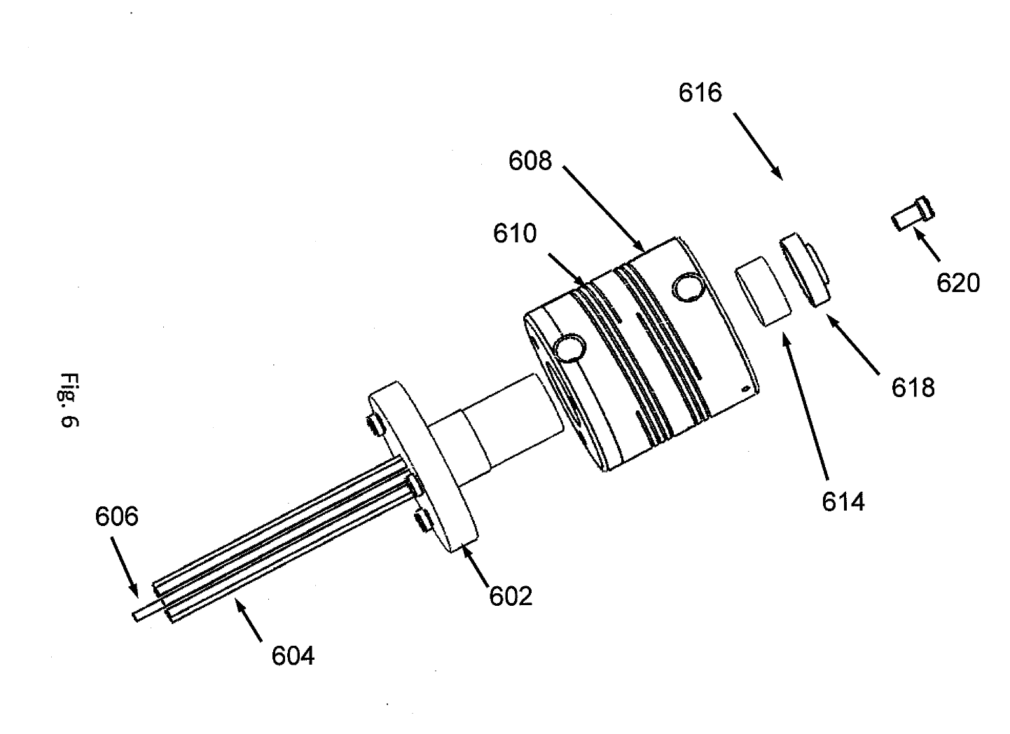

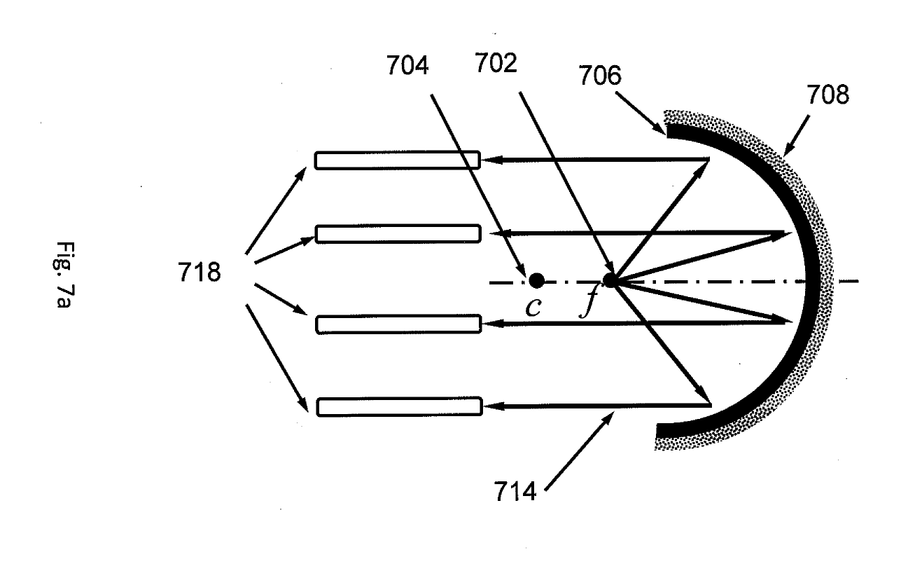

In one embodiment, a direct force feedback algorithm as shown in FIG. 4 controls a teleoperated needle placement system. As shown in FIG. 4a a two-port model, the master robotic device 400 is controlled by master controller 402 which translates motion commands from human operator 430 to slave robot 406 which is controlled by slave controller 404. The measured interaction force between needle and tissue in patient 423 are measured and transmitted through slave controller 404 to master robot 400 that display this force appropriately. In FIG. 4b, the commanded position signal 410 from master device 408 is translated to trajectory planner 412 that provides reference signal for slave controller 414. The slave robot 416 with integrated fiberoptic force sensor 418 provides the feedback force 420 which is scaled appropriately and fed into master device controller 422. Force or motion scaling may be used to increase precision, decrease hand tremor or vibration of motion commands provided by operator 430, or implement virtual fixtures or other guidance aids to help guide motion of the slave robot 416 in the patient 432.

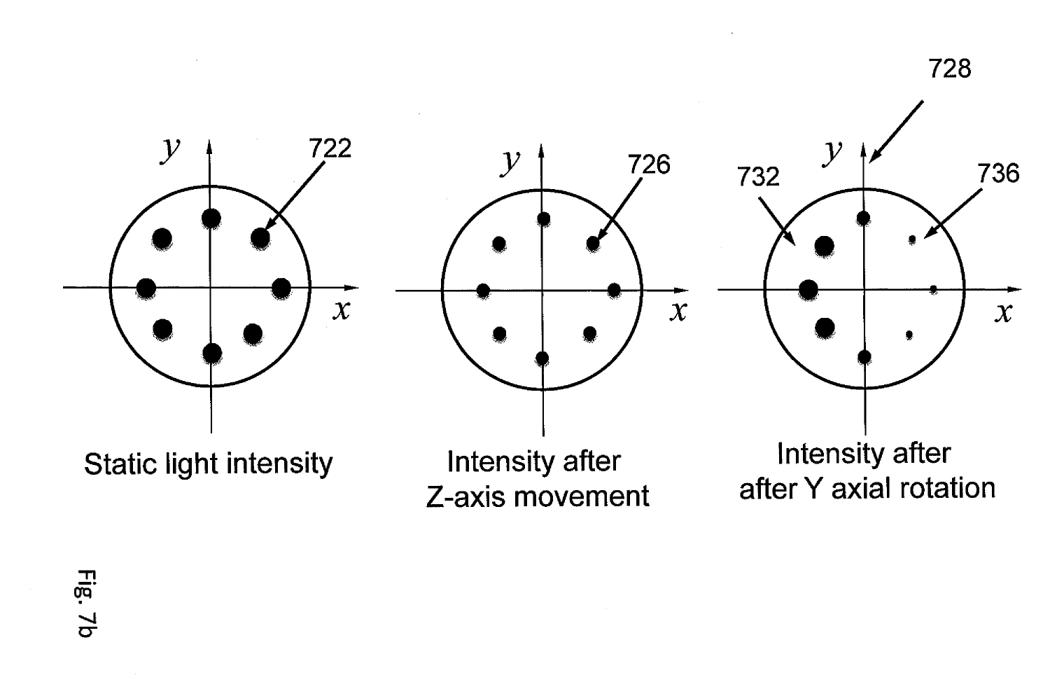

In one embodiment, the teachings are used to control percutaneous needle or other surgical tool insertion. A biopsy needle-like haptic gripper 502 as shown in FIG. 5 is used to assist heuristic and intuitive needle manipulation and attaches to a haptic master device at interface 504. In one embodiment, bracket 512 couples to interface 504 and supports control electronics 510 and gripper or handle 502. Buttons 506 and 516 couple to the circuit board 510 at 508. The circuit 510 and other components are enclosed in shell or cap 514. In alternate embodiment, other haptic grippers or handles may be used to mimic the surgical tool being manipulated by the slave robot. One embodiment of these teachings is intended to allow remote insertion of the tool from a remote location while maintaining the sensation of direct insertion. Remote may refer to immediately adjacent to the slave robot inside the MRI scanner room, a further location within the MRI scanner room, from within the MRI console or control room, from a doctor's office, or from any other on-site or off-site location.

Generally, the needle has 3-DOF Cartesian motion. In one embodiment, rotation of the needle about its axis is employed to improve the targeting accuracy and reduce insertion force. Alternatively, rotation may be used for active steering of the needle along a specified path or for correction of a path deviating from the target. Needle rotation may be controlled manually with or without haptic feedback. In one embodiment, needle rotation is controlled autonomously. In a further embodiment, needle rotation is autonomously controlled to steer the needle path to compensate for errors in needle placement. The needle may be steered or otherwise controlled based on the tip bevel angle, pre-curved cannulas or stylets, manipulation of the needle base, or other means. In an alternate configuration, the needle may be rotated continuously to minimize needle deflection during insertion. In one embodiment, needle rotation and translation can implemented to steer the needle using spatial duty-cycle based approach. The targeting error in Cartesian space can be used to determine the needle curvature using inverse kinematics. The ratio between this curvature over the maximum curvature is the input to trajectory planner that provides the control strategy between needle rotation angle and rotation velocity. The planned relationship between rotation position and velocity is an insertion velocity independent control that can steer the needle to target position by closed-loop control. The position information of the needle can be provided by optical-flow based tracking or other tracking and segmentation methods. Alternatively, needle tip position can be estimated using a series of MRI transverse needle void image slices, the known needle base position and needle length. Each transverse needle void image slice can be segmented to localize the position of needle void. According to the 3D information assimilated from the images, tip estimation can be posed as a boundary value problem for Euler-Bernoulli beam. Beam bending theory or spline minimization method can estimate the shape of needle in terms of minimum energy. In particular, thin plate spline can be used as basis function for representing coordinate mappings. Force sensing may be incorporated into the needle steering algorithm.

In one embodiment, one or more buttons or other user inputs 506 and 516 on the gripper are used to control the robot. In one configuration, the operator can push the first button 506 to start/stop the axial rotation of the needle and the second button 516 is used to fire the biopsy gun when it is in target position. Alternatively, the buttons can be used to select targets or to constrain the needle motion to 1-DOF insertion along the needle axis needle is appropriately aligned. In one embodiment, the robotic guide aligns the needle axis, and the needle is then inserted along that axis with force feedback using the master manipulator device. More generally, other buttons, switches, joysticks, or other input devices can be used to control many other modular and user-defined motions. The additional interfaces may be integrated into the haptic master device or in a separate device.

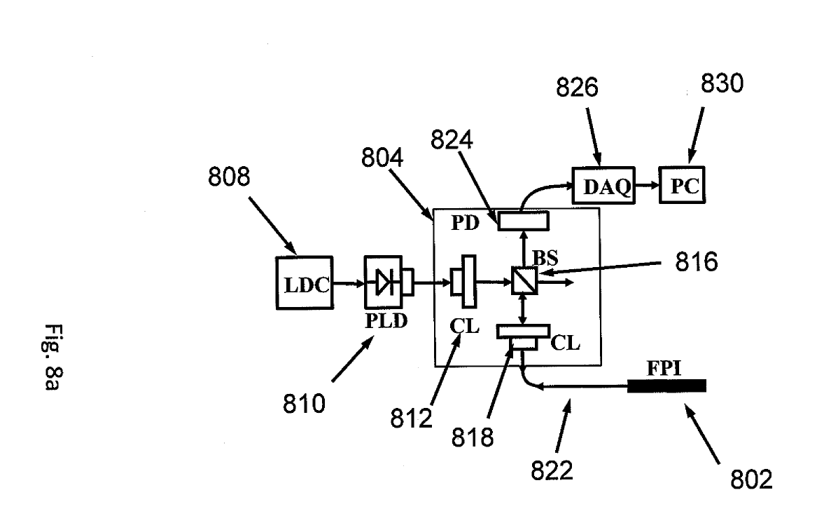

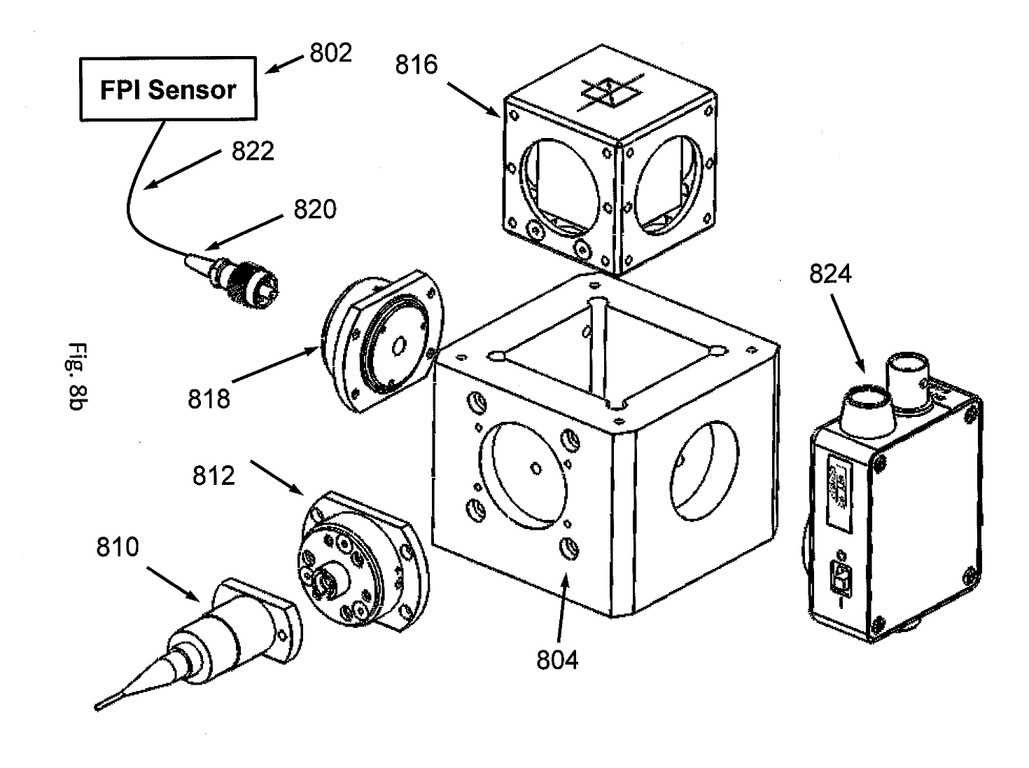

FIG. 6 shows an exploded view of one embodiment of an optical force sensor prototype. One embodiment of the sensor has four major components: the fiber holder 602 with eight 1 mm diameter through holes to arrange the receiver fibers 604 and an additional central hole for the emitter fiber 606, the flexure 608, the spherical mirror 614 and an adjustable mirror mount unit 616. The mirror mount unit 616 includes an adjustable bracket 618 and an adjustment screw 620. Adjustment screw 620 adjusts the position of mirror holder 618 to translate the mirror 614 to appropriately set the focal point. Other number of fibers, fiber sizes, fiber arrangements, and combinations of emitters and receivers may also be used.

The optical sensing mechanism in one embodiment shown in FIG. 6 is an economical and succinct structure which uses one spherical mirror 614 and multiple optical fibers 604 and 606. The incident light emitted along fiber 606 from a point source gets reflected by the front of spherical mirror 614, and the reflected light can be sensed by the tip of multiple optical fibers 604 which forms in circular pattern. The mirror 614 may be concave spherical or take on an alternate another shape. The top surface of flexure 608 flexes under an applied load, thus redistributing the emitted light over the receiver fibers 604.

Redundant measurements help to minimize the measurement uncertainty, signal drifting and environmental noise. Light intensity may be modulated to reduce the effect of ambient light and other external disturbances.

In one embodiment, the light signal emitted from a high-output infrared LED along fiber 606 is reflected by a 9 mm diameter concave spherical mirror 614. (It should be noted that the choice of light source, the dimensions used in this exemplary embodiment for the choice of components are not limitations of these teachings.) Alternatively, laser or other light sources may be used. The emitting position of the LED is designed to be within desired range of the focus point of the mirror, so the reflected light travels back to the emitting side with maximal intensity, where eight fiberoptic photodiodes with appropriate wavelength sensitivity are in circular pattern to detect the reflected light. The light is transmitted through the glass optical fibers 604 with 125 μm cladding diameter to the electronic board outside of the scanner room. All fibers are contained in a ribbon cable and conveniently couple to the controller through a single multi-fiber MTP connector. The glass fibers are inserted to the fiber holder whose inner holes are bonded with glue. The fiber jackets at the end of the fiber holder (3 mm long) are stripped off and tips are polished with fine sand papers to maximize the received light.

The response of these optical sensors as a function of the distance to the mirror has two segments: the first linear and sensitive segment in the range below 0.6 mm, and a low and decreasing sensitive segment for the higher range above 1 mm. Since a linear response is desired, in one embodiment the sensing part is kept to be within the first segment of the response curve. To guarantee high sensitivity and linearity of the sensor, the flexure deflection should be kept within the linear response segment in both directions. It is also desirable to have small deflection to obtain high stiffness and bandwidth. A plastic screw 620 is used to fix the mirror bracket 618. The simple and accurate adjustment structure can translate the mirror 614 into/out of body of the flexure 608. The dimension of one embodiment of the sensor is 36 mm in height and 25 mm in diameter and it weights 36 g equipped with 4 m optical fiber for scanner room communication.

Alternate fiber types, mirror types, light sources, light receivers, and connectors may be used and are part of these teachings. In a further configuration, the LED or laser light sources and the photodiodes or other photodetectors are located on a circuit board attached directly to component 602. Light guides or short fibers may be used, or the light may be directly transmitted to the mirror and reflected directly onto the photodetectors. In a further configuration, a position sensitive detector (PSD), CCD, or other multi-element photodetector may be utilized to determine the change in light distribution reflected from mirror 614.

Redundant measurements help to minimize the measurement uncertainty, signal drifting and environmental noise. Light intensity may be modulated to reduce the effect of ambient light and other external disturbances.

The design of flexure 608 is configured to provide force and torque sensitivity in the desired directions while minimizing effects of other forces and torques. One embodiment of the flexure 608 is capable of sensing axial force and lateral torques with high accuracy while tolerating off-axis forces and torques. Two parallelogram-like segments 610 of helical circular engravings in the structure have intrinsic axial/lateral overload protection capability and minimize the effects of lateral forces and axial torques. Other flexure designs can be used for other desired fore/torque combinations. The structure of the sensor is simple and facilitates fast and low cost manufacturing. The flexure is compact and simple, and it allows simpler fiber cables and electronics.

Transverse sensitivity is an important design factor of force sensor. To achieve minimal transverse sensitivity, it is preferable for the flexure to be stiffer to the forces applied in the other directions. The flexible hinges structure in this design with low thickness-to-width ratio would generate good direction selective stiffness. A novel flexure mechanism was designed and the finite element analysis was performed to aid the optimization of the design parameters. The flexure converts the applied forces and torques into displacement of the mirror thus generating a light intensity change. The structure should be simple to facilitate machining process. In order to guarantee measurement isotropy, a cylinder structure with engraved elastic curves was used in one embodiment. In one configuration, the flexure is machined using traditional machining processes. Alternatively, the sensor may be molded. One configuration of the sensor is single use and disposable. In one embodiment, the flexure structure may be constructed from rigid, MRI-compatible materials that are suitable for common sterilization practices used in a hospital setting. Building materials include, but are not limited to high strength plastics (including PEEK and polyetherimide), aluminum alloys, composites, ceramics, titanium alloys, etc. By implementing materials such as these, the image quality of the scanner can be preserved, allowing the user to take full advantage of in situ image guidance. In one configuration, the sensor is entirely non-metallic. Transverse sensitivity is an important design factor of force sensor. To achieve minimal transverse sensitivity, it is preferable for the flexure to be stiffer to the forces applied in the other directions. The flexible hinges structure in this design with low thickness-to-width ratio would generate good direction selective stiffness.

In one embodiment, although not limited thereto, the haptic system comprises an MRI-compatible force sensor which is designed for monitoring forces in the 0-20 Newton range with a sub-Newton resolution. In one configuration of the present teachings, the fiberoptic sensor enables 2-DOF torque measurement and 1-DOF force measurement.

One representative application of the 3-axis force/torque sensor with this range and resolution is for interventional procedures including needle biopsy and brachytherapy inside the MRI scanner. This configuration may be ideal for other needle-based procedures in MRI both with and without robotic assistance. Other configurations may be used for other applications. In one configuration, the fiberoptic sensor is used as a joystick to control a robot motion or interface with software. In another configuration, the sensor is used for rehabilitation or functional imaging studies. One embodiment of this sensor provides 3-DOF force measurement in percutaneous prostate interventions in 3 Tesla closed-bore MRI. Additional applications include other field strengths, open and closed bore MRI scanners and other surgical procedures including needle/electrode insertion during deep brain stimulation and needle based liver ablation. These do not represent the entirety of the potential applications.

One representative application of the 3-axis force/torque sensor with this range and resolution is for interventional procedures including needle biopsy and brachytherapy inside the MRI scanner. This configuration may be ideal for other needle-based procedures in MRI both with and without robotic assistance. Other configurations may be used for other applications. In one configuration, the fiberoptic sensor is used as a joystick to control a robot motion or interface with software. In another configuration, the sensor is used for rehabilitation or functional imaging studies.

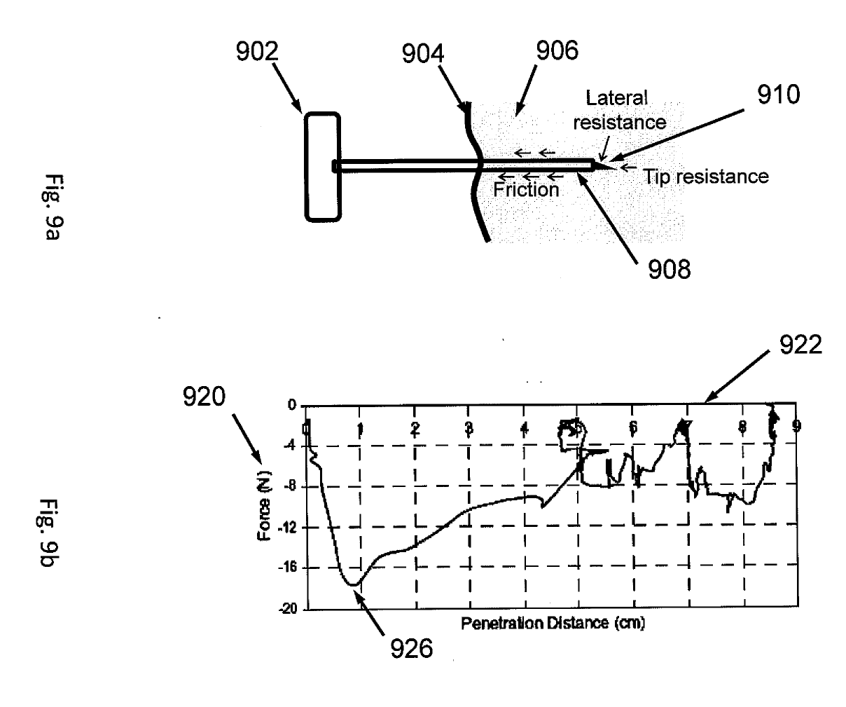

In one embodiment of the optical force sensor, a point source is assigned in the focal position 702 as shown in FIG. 7a, the reflected light from the front concave surface 706 of mirror 708 is parallel to the optical axis 704 therefore engender maximal light received by the fibers. As shown in FIG. 7b, if the relative axial distance between the light source and mirror increases, there would be proportional light intensity decrease from 722 to 726 in all of optical fibers which can be monitored by the interface electronics. If the mirror rotates along the tangential axes such as 728, there will be an asymmetric light intensity variance between 732 and 736 in the fibers, which can be detected by the interface electronics.

In one embodiment, the number of receiving fibers 718 in this design is 8, but the minimum number required for this sensor structure is 3. The simple mechanical structure of the flexure allows the deployment of more fibers which guarantees robust, high-fidelity force sensing capability. The fibers may all be at the same distance from the center, or they may be arranged in another configuration. The emitter may be in the center with receivers at the outside. Alternatively, there may be multiple switched or otherwise distinguished emitters with one or more receivers.

Redundant measurements help to minimize the measurement uncertainty, signal drifting and environmental noise. Light intensity may be modulated to reduce the effect of ambient light and other external disturbances.

In one calibration process, the sensor is mounted on a vibration isolating optical table using designed fixtures. Calibrated brass weights incrementally apply 100 g axial forces (up to 9.8 Newton) on the sensor. The 8 channel voltage outputs are recorded for 10 seconds for each configuration. The corresponding recorded voltage values were averaged to get the mean voltage output for each channel. The same procedure was performed to decreasingly unload the weight to evaluate hysteresis. Alternative calibration processes include using shape from motion techniques.

By taking advantage of this force sensor, the in-vivo insertion force can be monitored, but alternatively, this system can take advantage of it to perform active force control during the insertion procedure. Active force control and monitoring would provide high fidelity surgery and reduced operational time. The sensor can be used to measure tissue interaction forces with electrode tip or needle shaft and tip, detection of obstructions, guidance for steering needle/electrodes, and provide a sensing input for a cooperatively controlled robot, input for functional neurology studies, rehabilitation device.

One specific application is a semi-automated needle guide for MRI-guided prostate brachytherapy and biopsy with haptic feedback. Additional uses include a generic multi-axis force/torque sensor to monitor surgical intervention force or the human grip force during neural rehabilitation or other purposes. The sensor may also have applications in environments where electronics cannot be tolerated, i.e. industrial, dangerous and explosive environments and explosion prevention environment.

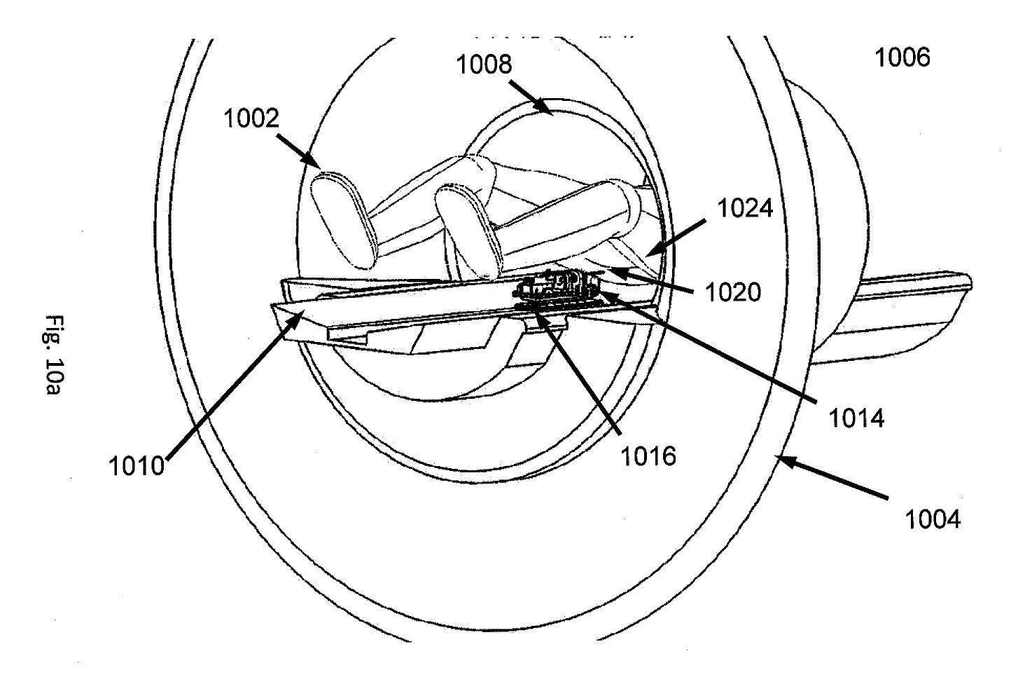

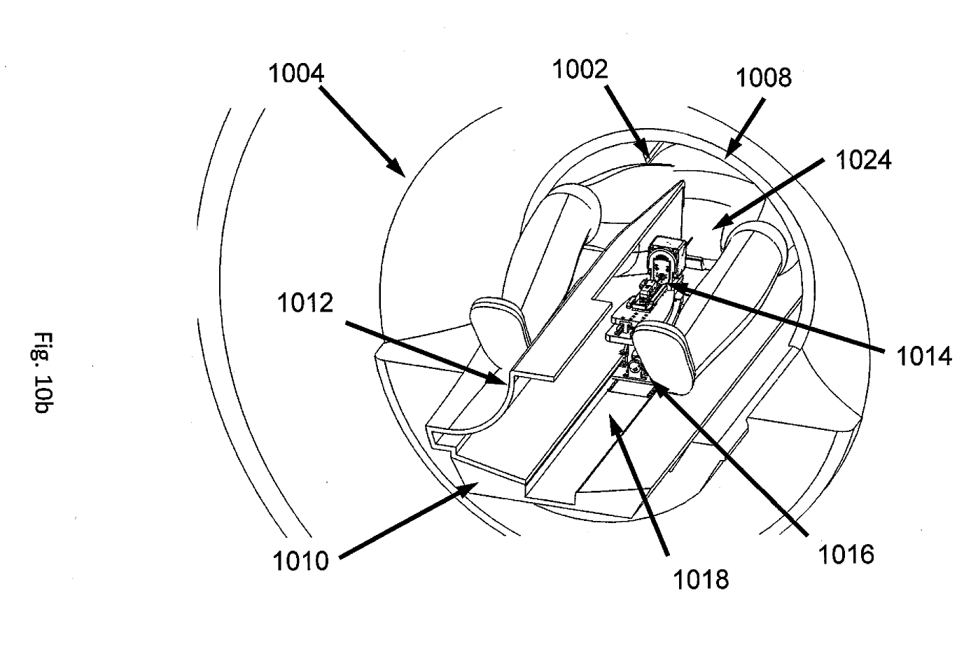

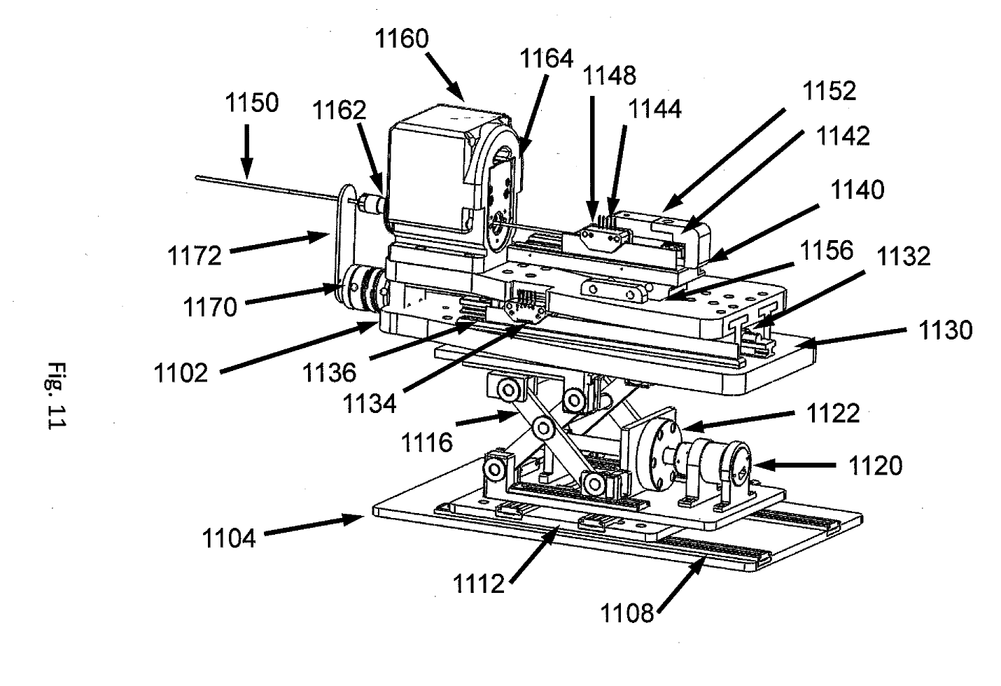

Alternate embodiments of fiberoptic force sensing in MRI can be implemented using wavelength-modulated methods including Fiber Bragg grating (FBG) or phase modulated method including Fabry-Perot interferometer (FPI) based strain sensing (see, for example, Yoshino, T., Kurosawa, K., Itoh, K., Ose, T., Fiber-Optic Fabry-Perot Interferometer and its Sensor Applications, IEEE Transactions on Microwave Theory and Techniques, Volume: 30 Issue: 10, October 1982, pp. 1612-1621, and U.S. Pat. No. 6,173,091, both of which are incorporated by reference herein in their entirety for all purposes). The present teachings include a miniature fiberoptic force sensor to measure needle insertion forces in MRI-guided prostate interventions. In one embodiment shown schematically in FIG. 8a, a 1-DOF FPI sensor is capable of measuring axial needle insertion force in a similar mechanical setting of strain gauges. FIG. 8b shows an exploded view of one embodiment of the FPI sensor. The FPI sensor 802 acts as an optical strain gauge which is incorporated into the slave robot or master device and couples to the sensor interface 804 though fiber 822 and connector 820. Interface 804 may be inside the robot controller or acts as a standalone interface. Light source 810 is a laser diode controlled by laser diode controller 808. Optical alignment interface 812 aim the laser light into beam splitter 816. Light from the beams splitter is again aligned by optical alignment interface 818 to focus the light into fiber 822. The laser light reached FPI sensor 802 and the reflected light passes back though alignment interface 818 and beam splitter 816. An interference pattern is generated based on the strain induced in the sensor 802 which is incident upon photodetector 824. Photodetector 824 may be a photodiode focused on a specific location whose sensed intensity varies as the interference patter changes. The signal form the photodetector 824 is conditioned and read by data acquisition interface 826 and coupled to a PC, robot controller, or other device 830.

In an alternative embodiment, FPI optical strain gauges or Fiber Bragg grating strain gauges are embedded into a flexure. In one embodiment of these teachings, they are configured to measure 3-DOF forces or torques.

The fiberoptic force sensor embodiments in these teachings the sensor may be directly connected to the robot controller or another sensor interface inside the MRI scanner room. In an alternate embodiment, the fibers are passed out of the MRI scanner room and coupled to a standalone sensor interface or other sensor interface outside the scanner room.

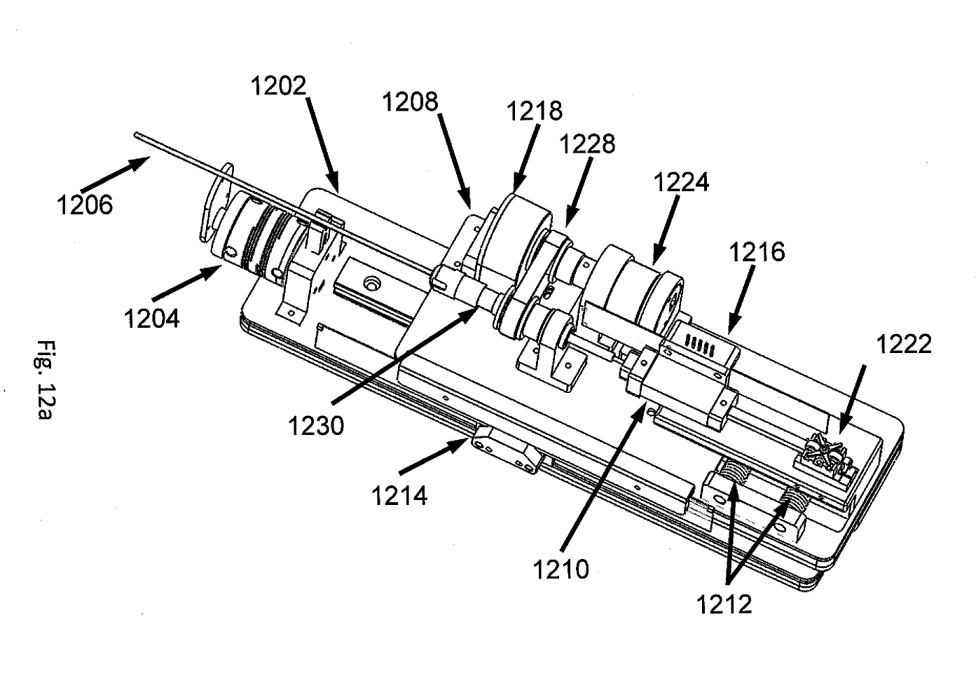

FIG. 9a depicts forces interacting on a needle during insertion. Needle 902 punctures skin or other tissue interface 904 and is inserted into tissue 906. The forces include axial forces along the needle axes and friction forces along the surface 908 of the needle. Forces present on an asymmetric bevel tip 910 will cause the needle to deflect during insertion. In one embodiment, we use these forces to actively control the needle insertion path. In a further embodiment, interactive MRI imaging is used to perform closed loop control of needle insertion. A further embodiment of the present invention uses force information sensed during the needle insertion for classification of tissues. In one configuration, needle forces and MRI imaging are utilized together to classify tissue by type or pathology. Further, forces may be used in conjunction with anatomical imaging for assisting in localization of the needle tip. One configuration of such integrated sensors is one or more FPI of FBG fibers along the needle to measure needle bending and shape. In an alternate embodiment, sensing integrated into the needle is used for localizing the needle and control. A further embodiment of the present invention uses force information sensed during the needle insertion for classification of tissues. In one configuration, needle forces and MRI imaging are utilized together to classify tissue by type or pathology. Further, forces may be used in conjunction with anatomical imaging for assisting in localization of the needle tip.

FIG. 9b shows a representative plot of axial needle insertion force 920 as a function of penetration depth 922 (see, for example, Y. Yu, T. Podder, Y. Zhang, W. S. Ng, V. Misic, J. Sherman, L. Fu, D. Fuller, E. Messing, D. Rubens, J. Strang, and R. Brasacchio, “Robot-assisted prostate brachytherapy,” Medical Image Computing and Computer-Assisted Intervention—MICCAI 2006. 9th International Conference. Proceedings, Part I (Lecture Notes in Computer Science Vol. 4190), (Berlin, Germany), pp. 41-9, Springer-Verlag, 2006, which is incorporated by reference herein in its entirety for all purposes)). When the needle punctures the skin or other tissue interface, such as the capsule of the prostate, a peak in insertion force 926 is present. The present teachings are capable of sensing the insertion forces, including peaks in insertion force at tissue interfaces, and reflecting them back to an operator using a haptic master device.

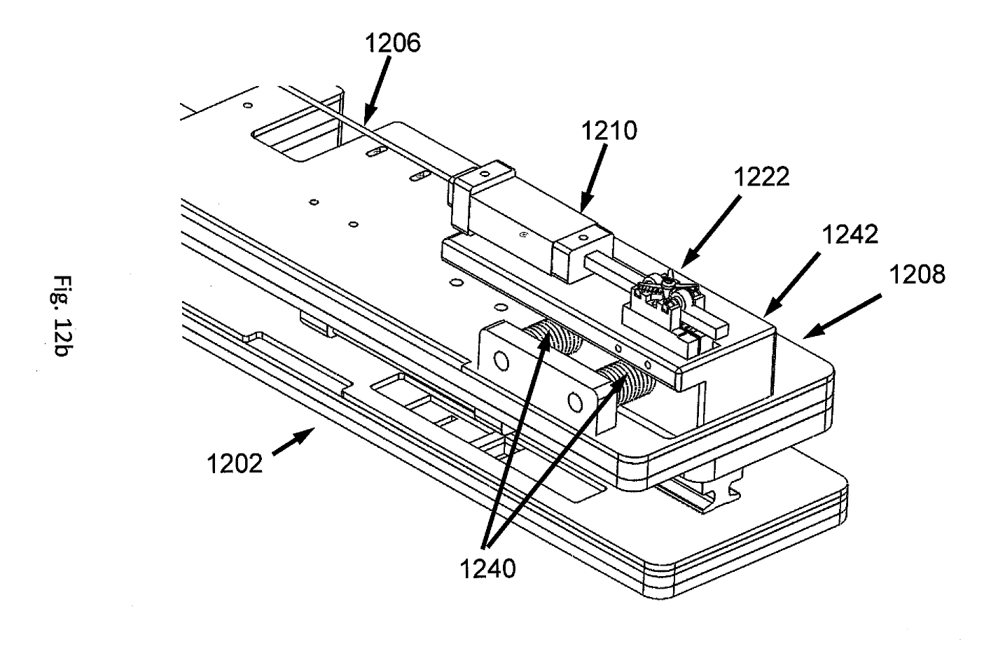

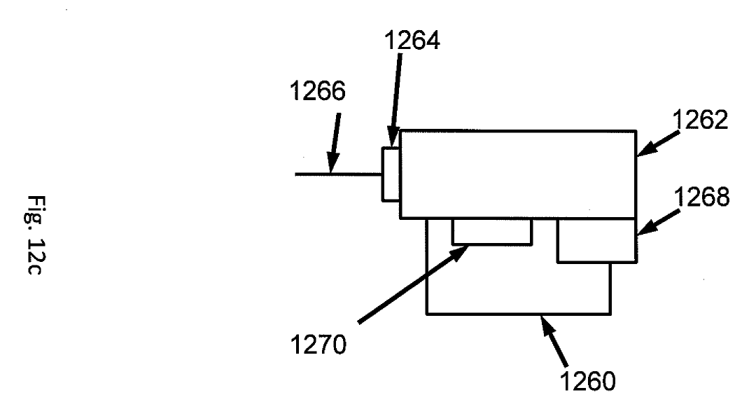

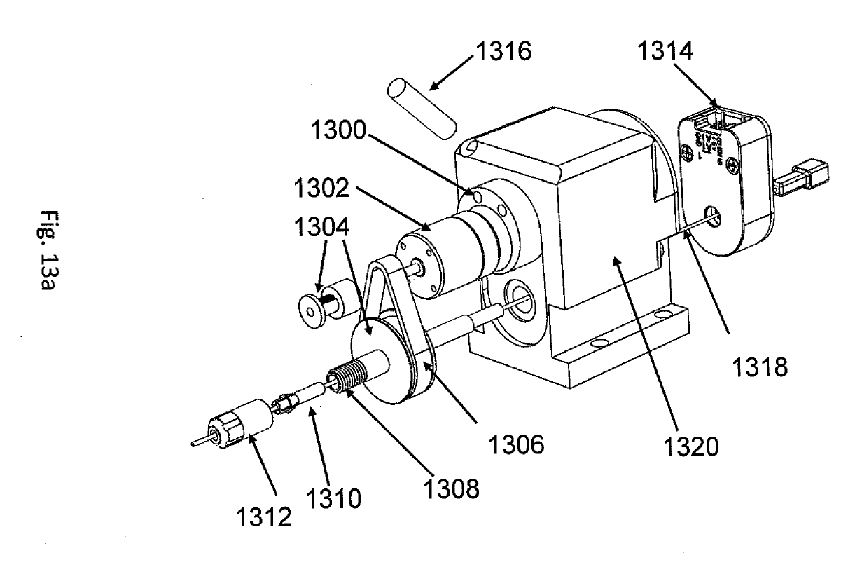

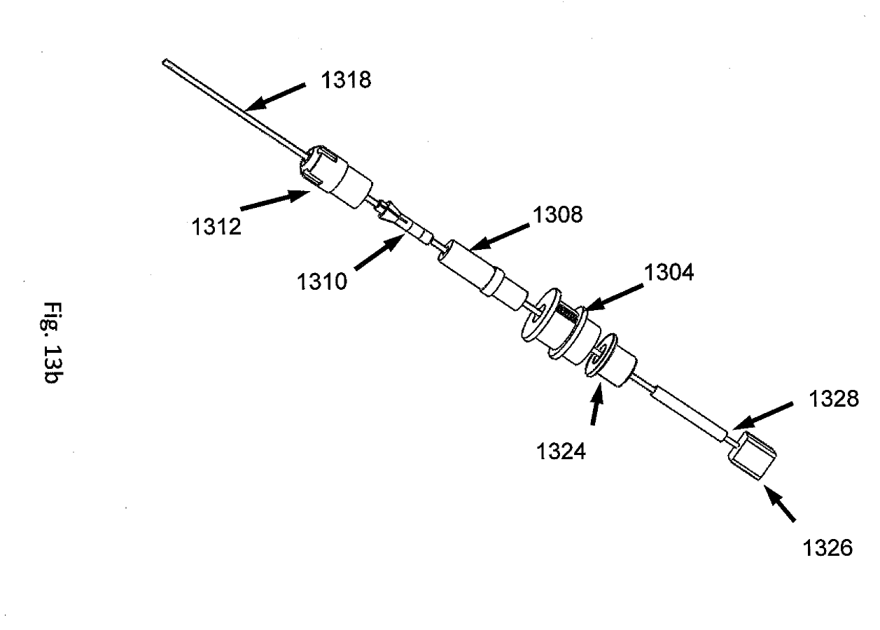

FIG. 10a depicts an embodiment of these teachings in which a needle insertion robot resides in the MRI scanner. The patient 1002 is located inside the MRI scanner 1004 which resides in MRI scanner room 1006. During imaging and an image-guided surgical intervention, patient 1008 is inside the MRI scanner bore 1008 on the bed, table, or couch 1010. An MRI-compatible robotic device 1014 is place inside bore 1008 of scanner 1004. Robot 1014 sits on base 1016. In one embodiment, the robot 1014 is a slave manipulator in a teleoperated system. In a further embodiment, robot 1014 controls placement of needle or surgical tool 1020 based in whole or in part by the motion of a haptic master controlled by the operator. One application of the present teachings is for prostate interventions including diagnosis with biopsy and treatment with brachytherapy. In these applications, needle 1020 is inserted into the entry point 1024 of the patient 1008 while acquiring real-time or interactive MRI image updates from MRI scanner 1004. In one embodiment, robot 1014 performs transperineal prostate needle placement through the patient's perineum 1024. FIG. 10b depicts a further embodiment wherein the robot 1014 consists of a needle driver module on Cartesian base 1016. The base 1016 sits on slide 1018 for inserting and removing the robot from the operative field. In one embodiment, the robot resides within a leg rest or tunnel 1012 that fits inside scanner bore 1008 One embodiment of the robotic needle placement device is shown in FIG. 11. The apparatus of one embodiment includes a MRI-compatible needle placement robot is actuated by piezoelectric actuators and used for prostate brachytherapy and biopsy. In one configuration, An MM-compatible modular 3-DOF needle driver module 1102 coupled with a 3-DOF Cartesian motion platform 1104. In one application, the device is a slave robot to precisely deliver radioactive brachytherapy seeds under interactive MM guidance.

One embodiment of the Cartesian motion platform 1104 contains 3-DOF motion. Linear slide 1108 provides motion along the axis of the scanner and linear slide 1112 provides lateral motion with respect to the scanner. Both axes 1108 and 1102 are actuated by linear piezoelectric ceramic motors and position is sensed by optical encoders. Alternate embodiments may use other joint encoding sensors including fiberoptics and linear potentiometers. Vertical motion mechanism 1116 is actuated by rotary piezoelectric motor 1120 through lead screw 1122.

One embodiment of the needle drive module 1102 provides 3-DOF motion including cannula rotation and insertion (2-DOF) and stylet translation (1-DOF). The independent rotation and translation motion of the cannula can increase the targeting accuracy while minimize the tissue deformation and damage. The module sits on platform 1130 that mounts to base stage 1104. Linear motion is provide along linear slide 1132 by piezoelectric motors. Joint position is sensed by optical encoder 1134 which reads encoder strip 1136. The inner stylet of the needle is controlled independently of the outer cannula by module 1140. Motor 1142 translates the stylet relative to the needle and encoder 1144 measures position. The hub 1148 of needle 1150's stylet contacts interface component 1152. Interface 1152 pushes the stylet hub 1148 relative to needle 1150. In one embodiment, interface 1152 incorporates force sensing for the axial needle insertion force. Needle rotation module 1160 allows for rotation of the needle about its axis as it is driven into the tissue. In one embodiment, module 1160 also includes tracking fiducials for locating the robot inside the MRI scanner to assist in registration and control. Module 1160 include a rotary piezoelectric motor that turns collect or needle clamp 1162 which is mechanically coupled to needle 1150. Encoder 1164 measure needle rotation. A force sensor 1170 couples to needle 1150 through interface 1172. One embodiment of force sensor 1170 is described in FIG. 6. Sensor 1170 measures lateral forces on the needle at or near the skin entry point. In an alternate embodiment, Sensor 1170 is integrated into interface and needle guide 1172. In a further embodiment, all of the needle or tissue contacting components are removable and either sterilizable or single use. Single use components in one embodiment of these teachings include interface 1172 and collet with guide tube 1162.

An embodiment of the needle driver module 1102 provides for needle cannula rotation, needle insertion and cannula retraction to enable the brachytherapy procedure with the preloaded needles. The device mimics the manual physician gesture by two point grasping (hub and base) and provides direct force measurement of needle insertion force by fiberoptic force sensors. To fit into the seamier bore, the width of the driver is limited to 6 cm and the operational space when connected to a base platform is able to cover the perineal area using traditional brachytherapy 60 mm×60 mm templates. The robot maximizes the compliance with transperineal needle placement, as typically performed during a TRUS guided implant procedure. This design aims to place the patient in the supine position with the legs spread and raised with similar configuration to that of TRUS-guided brachytherapy.

In further embodiment of these teachings, the following mechanisms are implemented to minimize the consequences of system malfunction. a) Mechanical travel limitations mounted on the needle insertion axis that prevents linear motor rod running out of traveling range; b) Software calculates robot kinematics and watchdog routine that monitors robot motion and needle tip position; and c) Emergency power button that can be triggered by the operator. The robot components of one embodiment are primarily constructed of acrylonitrile butadiene styrene (ABS) and acrylic. Ferromagnetic materials are avoided. Limiting the amount of conductive hardware ensures imaging compatibility in the mechanical level. In one configuration, only the needle clamp and guide (made of low cost ABS plastic) have contact with the needle and are disposable.

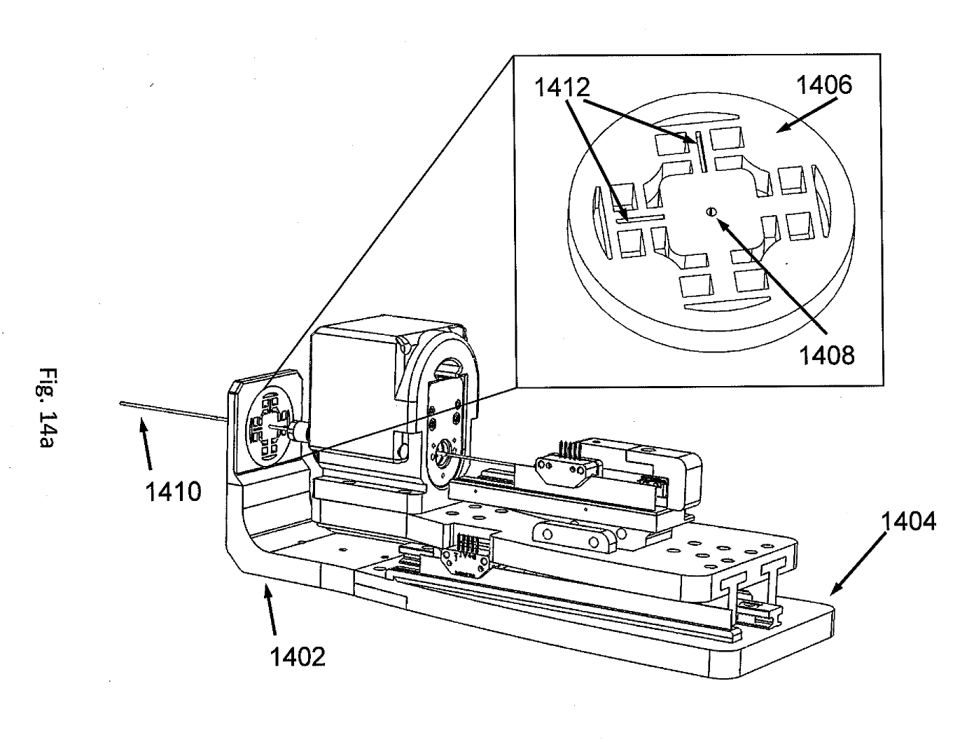

During needle placement procedure, to accomplish needle insertion, a needle can be mounted on the slave robot. For one embodiment, the slave robot can have 4-DOF which provides the 1-DOF needle translation and Cartesian base positioning. One embodiment of the needle drive module 1202 shown in FIG. 12a incorporates 3-DOF in addition to a Cartesian base. A force sensor 1204 can be coupled with the needle 1206 to provide direct needle force measurement. Sensor 1204 can provide lateral forces and a 1-DOF sensor 1210 provides axial force sensing. Optical encoder 1214, 1216, and 1218 measure the position of each of the 3-DOF on the driver module. Actuator 1222 drives the stylet of needle 1206 with respect to the cannula which is attached to base 1208. A further linear actuator drives base 1208 and needle 1206 with respect to the base 1202 which is attached to the 3-DOF Cartesian base. Rotary actuator 1224 drives a collet 1230 through belt 1228. This allows the needle 1206 to be clamped into collect 1230 and have precisely controlled rotation angle. In one embodiment, the actuators 1222 and 1224 are piezoelectric motors.

Once a needle, preloaded brachytherapy needle, or biopsy gun is inserted into collet 1230, the collet can rigidly clamp the outer cannula shaft 1206. In the case of a solid needle, guide wire or other instrument for insertion, the collet 1230 clamps onto needle 1206 and there is no differentiation between inner stylet and outer cannula. Since the linear motor 1222 is collinear with the collet and shaft, an offset must be induced to manually load the needle. The apparatus shown in FIG. 12b represents one embodiment of needle loading mechanism. The mechanism includes a brass spring preloaded mechanism 1240 that provides lateral passive motion freedom. The operator can squeeze the mechanism and offset the top motor fixture 1242 then insert the needle 1206 through plain bearing housing and finally lock with the needle clamping. This structure allows for easy, reliable and rapid loading and unloading of standard needles.

FIG. 12c illustrates a block diagram of an embodiment of a slave robot. As shown in the block diagram FIG. 12c, the needle driving module 1262 which is driven by MRI-compatible actuating component, resides on base component 1260. The base component motion is measured by a sensing component 1270. The force sensing component 1264 measures the needle insertion force along the needle 1266.

By actively steering and inserting needles, the needle can target 3D position and the force measurement threshold would avoid non-soft tissue interaction. To compensate for the needle deflection, the needle could be axially rotated. The needle deflection estimation algorithm can be used to find the appropriate insertion depths at which needle rotations are to be performed.

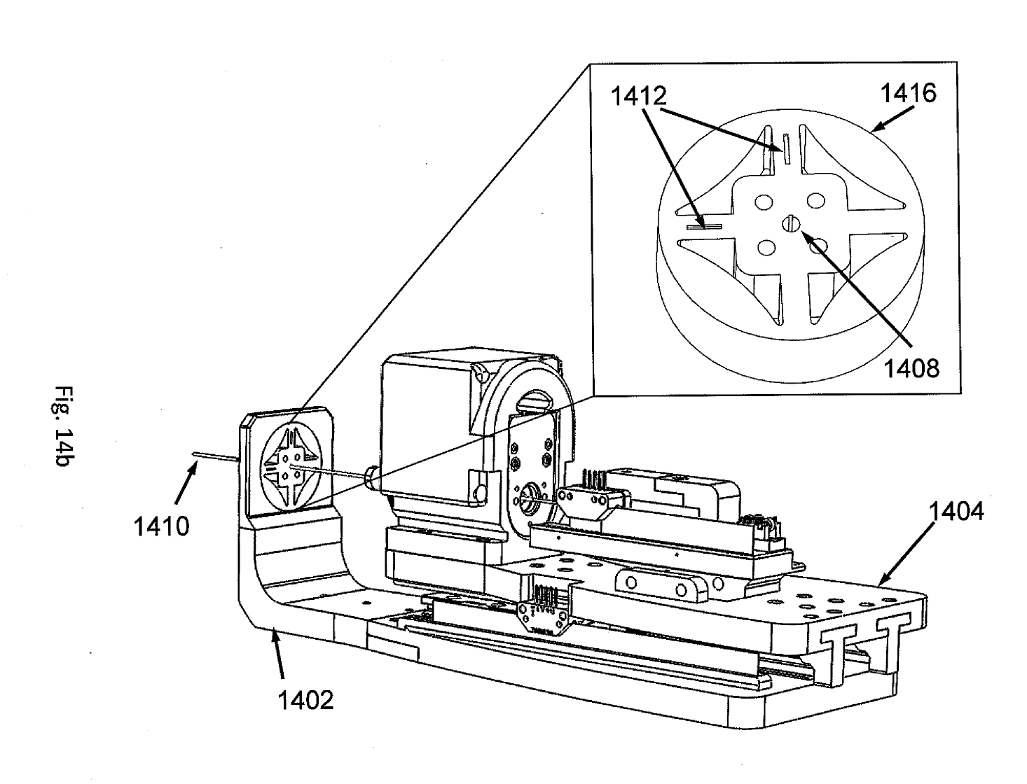

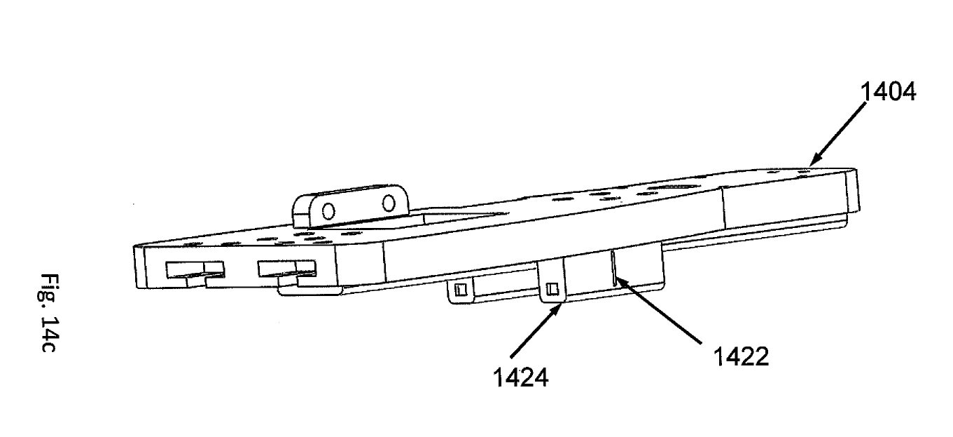

FIG. 13a shows one embodiment of the needle clamping and rotation apparatus. Needle 1318 is clamped to collet sleeve 1308. Pulleys 1304 and belt 1306 mechanically couple sleeve 1308 to rotary actuator 1302. Eccentric tensioner 1300 tightens belt 1306. Encoder 1314 precisely measures needle rotation angle.

Dynamic global registration between the robot and scanner is achieved by passive tracking the fiducial frame 1320 in front of the robot. The rigid structure of the fiducial frame is made of ABS and seven MRI fiducials 1316 are embedded in the frame to form a Z shape passive fiducial. Any arbitrary MR image slicing through all of the rods provides the full 6-DOF pose of the frame, and thus the robot, with respect to the scanner. Thus, by locating the fiducial attached to the robot, the transformation between the patient coordinate system (where planning is performed) and that of the needle placement robot is known. To enhance the system reliability and robust, multiple slices of fiducial images are used to register robot position using principal component analysis method. The end effector location is then calculated from the kinematics based on the encoder positions.

The needle driver allows a large variety of standard needles utilizing a clamping device shown in FIG. 13b that rigidly connects the needle shaft 1318 to the driving motor mechanism. One embodiment of the needle clamping structure is a collet mechanism 1310, a hollow screw 1308, and a nut 1312 twisted to fasten the collet thus rigidly locks the needle shaft on the clamping device. In this embodiment, stylet 1328 and hub 1326 are fixed to the driver. In alternate embodiment, the outer cannula and inner stylet may both rotate together or independently. The clamping device is connected to the rotary motor 1302 through a timing belt 1306 that can be fastened by an eccentric belt tensioner 1300. The clamping device is generic in that a set of collets can accommodate a width range of needle diameters. The needle driver is designed to operate with standard MR-compatible needles of various sizes. The overall needle diameter range for one embodiment is from 25 Gauge to 7 Gauge. The collet sets can not only fasten brachytherapy needle (typically 18 Gauge), but also biopsy needles and most other standard needles instead of designing some specific structure to hold the needle handle.

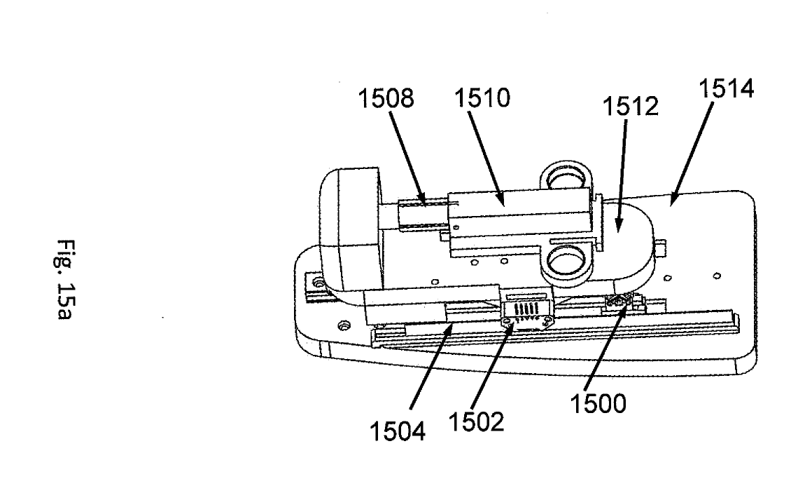

FIG. 14a illustrates one embodiment of a 2-DOF lateral force sensor coupled to the needle driver module. Needle guide 1402 attaches to needle driver module 1404. Sensor flexure 1406 has needle guide hole 1408 for needle 1410. Hole 1408 may have an insert to match various needle sizes or be sized for a specific needle diameter. The flexures in 1406 allow small amount of lateral 2-DOF motion to sense forces not mal to the needle axis. Strain sensors 1412 are integrated into the flexure. In one configuration, strain sensors 1412 are FPI sensors. FIG. 14b illustrates an alternate embodiment of a 2-DOF lateral force sensor coupled to the needle driver module. Sensor flexure 1416 is shown in an alternate configuration that contains strain sensors 1412. These are only two representative configurations. FIG. 14c illustrates one embodiment of a 1-DOF axial insertion force sensor coupled to the base of the needle driver module 1404. Strain in flexure 1424 represents axial forces applied to the motion stage form the motor. Strain sensor 1422 measures the strain in 1424 to reflect needle insertion forces. Alternative flexure designs 1406, 1416, and 1422 and strain sensor 1412 and 1422 types may also be utilized and are considered part of the present teachings.

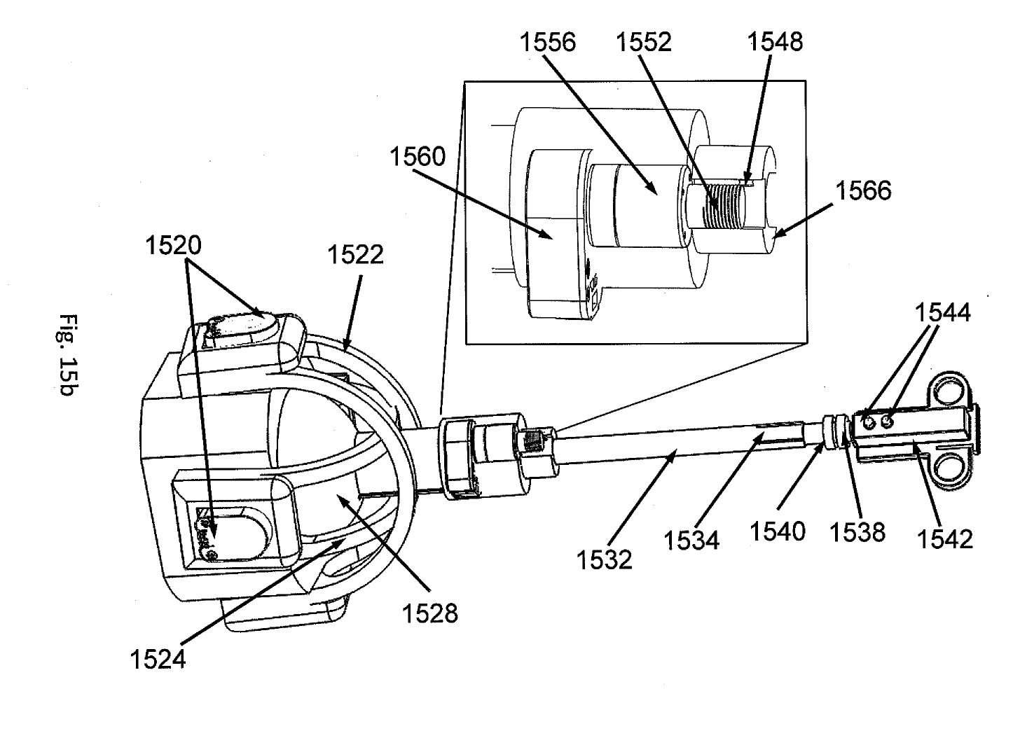

FIG. 15a illustrates an embodiment of a 1-DOF linear MRI-compatible haptic interface that serves as a master device. This is a representative embodiment of a haptic mater device for MRI-guided interventions and may take on other forms including additional degrees of freedom, and said linear degrees of freedom may be linear or rotary. An actuator 1500 is mounted on base plate 1514 and moves platform 1512. In one embodiment, actuator 1500 is a high stiffness piezoelectric linear actuator; in alternative embodiments it may take the foini of piezoelectric, pneumatic, hydraulic, electromechanical, or other actuation. Position sensor 1502 is mounted on the top plate 1512. In one embodiment, sensor 1502 is an optical encoder with linear strip 1504. In alternative embodiments, the sensor 1502 may be a reflective or through beam optical encoder, a potentiometer, a laser distance transducer, or other measurement means. An application-specific modular handle 1510 provides the user interface. The handle may be made to mimic the feel of a traditional tool. For example, handle 1510 demonstrates an embodiment for biopsy needle insertion. Force applied by a human operator on handle 1510 is measured by a sensor 1508. In one embodiment, sensor 1508 is a 1-DOF force sensor; in alternate embodiments, sensor 1508 may measure other DOF of forces and torques. In one embodiment of the system, force sensing is implemented as fiberoptic force sensing; alternatively forces and torques bay be measured by alternative means including but not limited to optical, resistive, capacitive, and piezoelectric sensors.

In one embodiment of the haptic device, the controller provides force feedback in an admittance control law where the force applied to handle 1510 is regulated in a closed loop controller using sensor 1508 and actuator 1500. The 1-DOF device may be used as a master haptic interface for needle insertion. In one embodiment, needle insertion force is sensed by a sensor on the slave robot or needle and that force is fed back to the operator through handle 1510. That force may be scaled to augment the user feedback experience. The operator applies force to handle 1510 which causes platform 1512 to move with respect to base 1514. Sensor 1502 measures the change in motion and commands the slave robot to follow. The bilateral teleoperator control scheme allows an operator to manipulate an MRI-compatible master from within the MRI scanner room and control the insertion of a needle with the sensation that they are manually performing the procedure. In a further embodiment, the operator only controls the motion in the insertion direction, and a robot controller autonomously controls additional DOF to control the needle trajectory and tip placement. In one embodiment, the robot controls the rotation of the needle during insertion to steer the needle tip based on forces applied to the beveled tip. Needle trajectory control may be used to automatically follow a predetermined path while the user only controls an insertion distance parameter. Alternatively, the needle path may be controlled to compensate for needle or tissue deformation based on models and or interactive image updates.

An alternative embodiment of a haptic interface in FIG. 15b. This embodiment represents one configuration of a multi-DOF MM-compatible haptic device where there is one active degree of freedom along the tool axis and three passive DOF. A large wrist arch 1522, a small wrist arch 1524 and a spherical joint 1528 provide pitch and yaw motion of the haptic device. The orientations of the joints are measured by sensors 1520 which may be optical encoders. An application-specific modular handle 1542 and function control buttons 1544 provide direct hand interface for an operator. A rotary position sensor 1540 which may be an optical encoder measures rotation of handle 1544. Rotational motion is transmitted by a bearing 1538 made of plastic, ceramic, glass or other compatible material. A rotary motor 1556, which in one embodiment is a piezoelectric motor, is fitted with a capstan drive that is used to guide the cable 1552 off the roller 1548 onto the flat side of a vertical shaft 1566. By rotating the motor, whose position is sensed by encoder 1560, the cable pulls either in or out producing a linear motion of handle 1542. To reduce friction, a precision ground shaft 1532 is used as the instrument shaft and glides inside two linear bearings. The bearings are slotted to allow a flat mounted to the shaft to protrude for the cable drive. Sensors 1534 can provide information about the forces and/or torques applied by the operator to handle 1544.

FIG. 15c depicts a block diagram of a master device. An MRI-compatible actuating component 1576 drives a base component 1578 whose motion is measured by an actuation sensing component 1580. A haptic interface 1570 connects to a force sensor 1572 and the top component resides on a motion carriage 1574. In one configuration, force sensor 1572 is an optical sensor that measures user interaction forces on handle 1570. A robot controller uses information from sensor 1572 to regulate the applied force by controlling actuating component 1576. The position sensed by sensor 1580 is used to control the position of a slave robot.

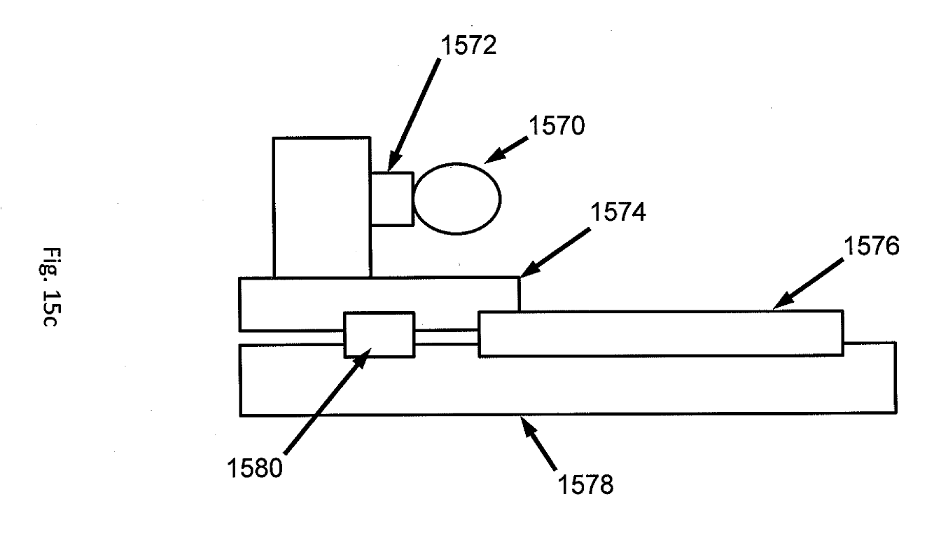

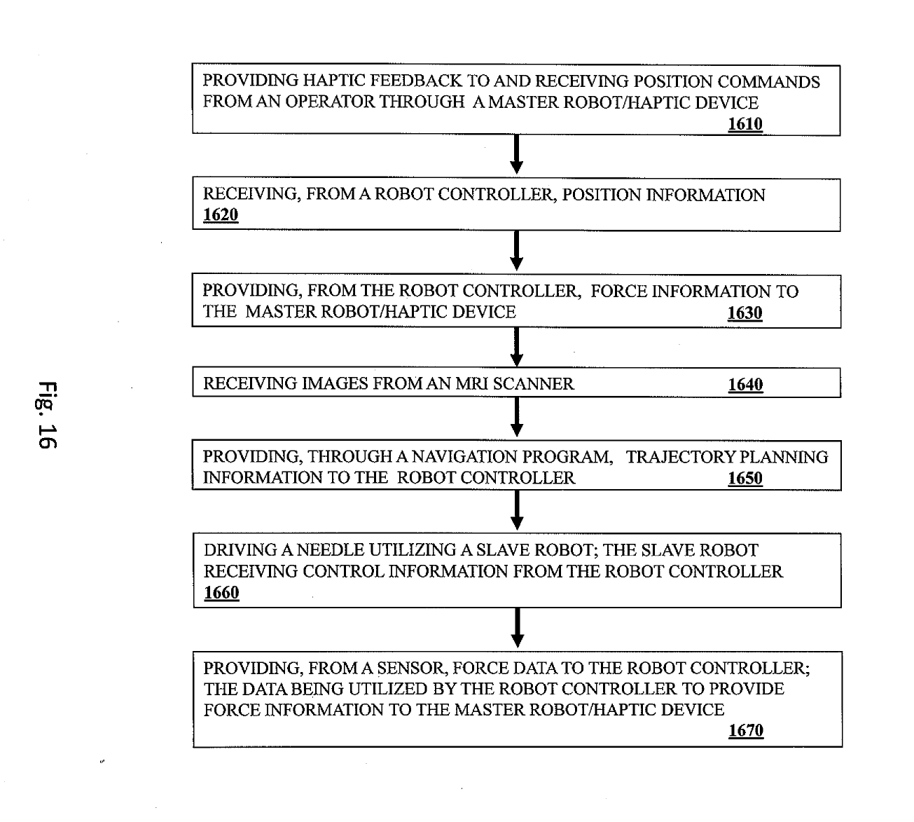

FIG. 16 illustrates one embodiment of the method for using the system of these teachings for performing MRI-guided interventional needle procedures. Referring to FIG. 16, the method includes the steps of providing haptic feedback to and receiving position commands from an operator through a master robot/haptic device (step 1610, FIG. 16), receiving, from a robot controller, position information (step 1620, FIG. 16), providing, from the robot controller, force information to the master robot/haptic device (step 1630, FIG. 16), receiving images from an MRI scanner (step 1640, FIG. 16), providing, through a navigation program, trajectory planning information to the robot controller (step 1650, FIG. 16), driving a needle utilizing a slave robot; the slave robot receiving control information from the robot controller (step 1660, FIG. 16), and providing, from a sensor, force and/or torque data to the robot controller; the data being utilized by the robot controller to provide force information to the master robot/haptic device (step 1670, FIG. 16). The method provides teleoperated force feedback and compensates for loss of needle tip force information.

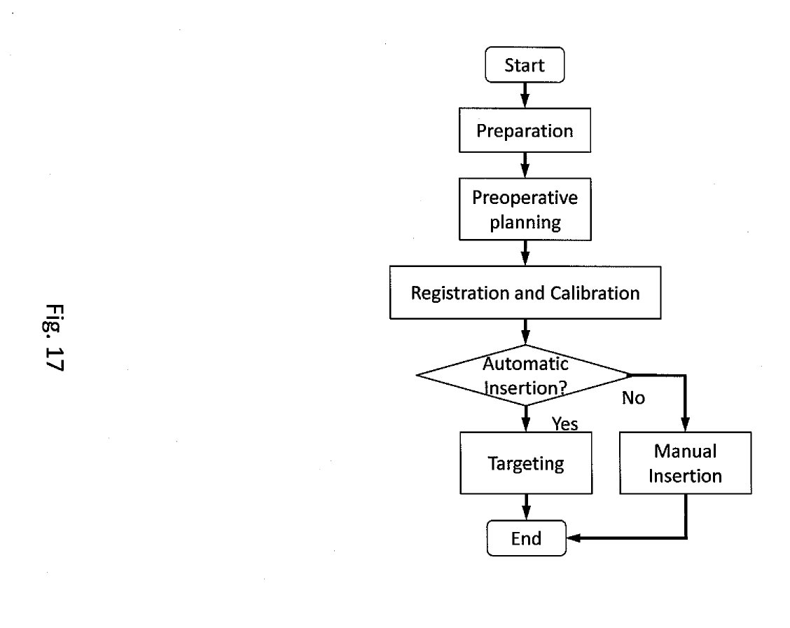

FIG. 17 illustrates one embodiment of the haptic assisted needle insertion work phases. It consists of patient preparation, preoperative planning, registration, calibration, automatic targeting or manual operation and emergency stop. The emergency stop is accomplished in mechanical and software design. The monitored force would be another quantity to enhance the operational safety and patient comfort. Automatic insertion refers to a fully automated needle insertion where the needle is actuated along a predefined path or using real-time imaging to guide the needle in a closed-loop motion. Manual insertion refers to the use of monitored forces fed back to a haptic mater device where an operator performs the needle insertion using the master device while the slave robot follows.

Briefly, one procedure incorporating the invention for MRI-guide transperineal prostate biopsy is described as follows:
1) Induce patient anesthesia and connect imaging coils and slide patient into scanner.
2) Place sterile insert into leg support tunnel and position leg support against perineum.
3) Drape robot base and attach sterile needle driver and load first biopsy needle.
4) Drive robot to initial configuration with retracted needle.
5) Acquire robot calibration images and pre-procedural images and registered to pre-operative data
6) Verify needle and robot trajectories.
7) Select entry point, and position and orient needle trajectory for current target.
8) Insert needle using haptic console. Rotate and steer needle if necessary.
9) Real-time imaging and fused navigation display and force monitoring.
10) Advance biopsy mechanism, followed by short imaging sequence to verify positioning against fused data set.
11) Fire biopsy gun.
12) Retract needle.
13) Disengage robot latch and slide robot to base of cradle.

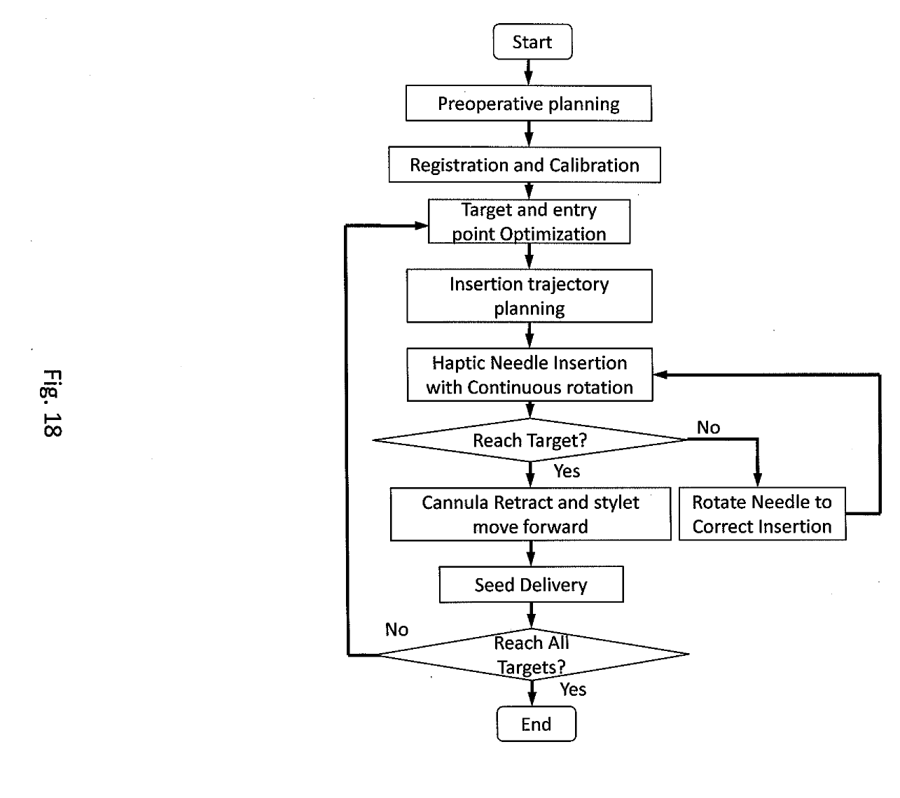

FIG. 18 illustrates a workflow corresponding to the use of one embodiment of the present teachings. The process starts with preoperative planning that may take place during the procedure, immediately prior to the procedure, or at an earlier time. The patient and the robot are located in the scanner and registered. A target and trajectory are defined in the patient images and located in the robot coordinate system. The needle or tool is inserted into the body to the target. The insertion may be under interactive or real-time MRI imaging. Alternate imaging modalities may also be used together or separately. The needle interaction forces are sensed by the needle driver module or by other means. The needle insertion forces may be reflected to a haptic mater controlled by the clinician. The needle may be rotated continuously to minimize deflection. Alternatively, the needle rotation may be controlled to steer or otherwise manipulate the needle insertion path. Closed loop control of needle placement using MRI images is included as part of the present teachings. In on embodiment, the needle insertion can be controlled by a haptic master and coordinated with semi-autonomous needle steering. In a semi-autonomous mode, real-time or interactive image updates provide information about at least one of the robot, needle, and target location. This information is used to actively iteratively guide the needle to the appropriate location utilizing a closed-loop controller. Needle localization may be in the form of tracked needles, instrumented needled, image based with the needle in a single imaging plane, or image based from cross sectional images of the needle. In one configuration, a limited set of cross-sectional images of the needle are acquired and used in conjunction with a needle bending model and information about the robot base location to determine the needle tip location and trajectory. Steering may be performed such that the operator only controls the depth parameter with force feedback while the robot controller automatically controls needle rotation or other DOF to compensate for misalignment with the target.

When the needle tip reaches the target, a secondary operation may be performed. In the case of biopsy, a biopsy gun may be fired and a tissue sample acquired. For brachytherapy seed placement using a preloaded needle, the cannula may be retracted to place the seeds. In one embodiment, a needle driver module's two linear motion stages move in a coordinated motion to place the seeds. This process is repeated for all needle insertions in the given procedure.

For the purposes of describing and defining the present teachings it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

While the present teachings have been described above in terms of specific embodiments, it is to be understood that they are not limited to these disclosed embodiments. Many modifications and other embodiments will come to mind to those skilled in the art to which this pertains, and which are intended to be and are covered by this disclosure. It is intended that the scope of the present teachings should be determined by proper interpretation and construction of the claims, as understood by those of skill in the art relying upon the specification and the attached drawings.

What is claimed is:

1. A system for MRI-guided interventional needle procedures, the system comprising:
a master device providing haptic feedback to and receiving position commands from an operator;
a robot controller receiving position information and providing force information to said master device;
a navigation component receiving images from an MRI scanner; said navigation component providing trajectory planning information to said robot controller;
a slave robot driving a needle; said slave robot receiving control information from the robot controller; and
a fiberoptic sensor operatively connected to said slave robot; said fiberoptic sensor providing data to said robot controller; said data being utilized by said robot controller to provide force information to said master device.

2. The system of claim 1 wherein the robot controller and the slave robot are compatible with the MRI environment.

3. The system of claim 1 wherein said fiberoptic sensor comprises:
a movable mirror mount structure;
a mirror mounted on said surface of said movable mirror mount structure;
a light providing optical fiber; said light providing optical fiber disposed along a direction of an optical axis of said mirror; said direction being determined substantially in the absence of motion of said movable mirror mount structure; one end of said light providing optical fiber providing light to said mirror; and
a plurality of light receiving optical fibers; said plurality of light receiving optical fibers being disposed along a periphery of said light providing optical fiber; said plurality of light receiving optical fibers being disposed such that, when torque is transmitted to said movable mirror mount structure, a substantially asymmetric distribution of light intensity is received at said plurality of light receiving optical fibers and, when force is transmitted to said movable minor mount structure, causing displacement along said direction of said optical axis, a substantially symmetric distribution of light intensity is received at said plurality of light receiving optical fibers.

4. The system of claim 3 wherein said mirror is a spherical mirror.

5. The system of claim 3 further comprising:
a flexure component comprising:
one end; said one end being operatively attached to a surface of said movable mirror mount structure;
another end disposed a distance away from said one end; and
an outer surface extending from said one and to said another end;
said outer surface comprising a plurality of flexures; a number of said plurality of flexures and dimensional characteristics of said plurality of flexures being selected to provide predetermined sensitivity to force and torque in predetermined directions; said force and torque being transmitted to said movable minor mount structure.

6. The system of claim 5 wherein said flexure component comprises MRI-compatible materials.

7. The system of claim 6 wherein said MRI-compatible materials are selected from high strength plastics, aluminum alloys, composites, ceramics, or titanium alloys,

8. The system of claim 3 wherein light provided by said light providing optical fiber is obtained from an infrared LED.

9. The system of claim 3 wherein light provided by said light providing optical fiber is obtained from a laser source.

10. The system of claim 3 wherein said plurality of light receiving optical fibers comprises at least three optical fibers.

11. The system of claim 1 wherein said slave robot comprises:
a base component;
an MRI-compatible actuating component moving said base component;
a first sensing component sensing motion of said base components; and
a needle driving module operatively disposed on said base.

12. The system of claim 11 wherein said MRI-compatible actuating component is a 3 degrees of freedom (3-DOF) MRI-compatible actuating component.

13. The system of claim 11 wherein said needle driving module comprises:
a stylet needle driving component comprising:
a stylet actuating component; and
a force sensing component; and
a cannula rotation component comprising:
a rotation actuating component; and
a rotation sensing component.

14. The system of claim 11 wherein said MRI-compatible actuating components comprise piezoelectric motors.

15. The system of claim 11 wherein said base and said needle driving component comprise:
a first platform;
a first linear actuating mechanism disposed on said first platform;
a first piezo-electric motor driving said first linear actuating mechanism;
a second platform disposed on said first linear actuating mechanism; said second platform being movable by said third linear actuating mechanism; and
a needle drive component mounted on said second platform; said needle drive component enabling needle insertion; the needle being operatively connected to the needle drive component.

16. The system of claim 15 wherein said MRI-compatible actuating device comprises:
a vertical motion mechanism disposed on said third platform; and
a second piezo-electric motor driving said vertical motion mechanism;
said first platform being disposed on said vertical motion mechanism

17. The system of claim 16 wherein said MRI-compatible actuating device further comprises:
a fourth platform;
a second linear actuating mechanism enabling motion in one direction on said fourth platform;
a third linear actuating mechanism enabling motion in a direction perpendicular to said one direction on said fourth platform;
a second piezo-electric motor driving said second linear actuating mechanism;
a third piezo-electric motor driving said third linear actuating mechanism;
said third platform being disposed on said second and third linear actuating mechanisms;
said third platform being movable by said third and second linear actuating mechanisms.

18. The system of claim 13 wherein said force sensing component comprises:
a flexure operatively coupled to said stylet driving component; said flexure configured and positioned such that axial forces induce strain in said flexure; and
a strain sensor sensing said induced strain.

19. The system of claim 16 wherein said strain sensor is a fiber-optic Fabry-Perot interferometer sensor.

20. A fiberoptic sensor comprising:
a movable mirror mount structure;
a mirror mounted on said surface of said movable mirror mount structure;
a light providing optical fiber; said light providing optical fiber disposed along a direction of an optical axis of said mirror; said direction being determined substantially in the absence of motion of said movable mirror mount structure; one end of said light providing optical fiber providing light to said mirror;
a plurality of light receiving optical fibers; said plurality of light receiving optical fibers being disposed along a periphery of said light providing optical fiber; said plurality of light receiving optical fibers being disposed such that, when torque is transmitted to said movable mirror mount structure, a substantially asymmetric distribution of light intensity is received at said plurality of light receiving optical fibers and, when force is transmitted to said movable mirror mount structure, causing displacement along said direction of said optical axis, a substantially symmetric distribution of light intensity is received at said plurality of light receiving optical fibers.

21. The fiberoptic sensor of claim 20 wherein said mirror is a spherical mirror.

22. The fiberoptic sensor of claim 20 further comprising:
a flexure component comprising:
one end; said one end being operatively attached to a surface of said movable mirror mount structure;
another end disposed a distance away from said one end; and
an outer surface extending from said one and to said another end;
said outer surface comprising a plurality of flexures; a number of said plurality of flexures and dimensional characteristics of said plurality of flexures being selected to provide predetermined sensitivity to force and torque in predetermined directions; said force and torque being transmitted to said movable mirror mount structure.

23. The fiberoptic sensor of claim 22 wherein said flexure component comprises MRI-compatible materials.

24. The fiberoptic sensor of claim 23 in wherein said MRI-compatible materials are selected from high strength plastics, aluminum alloys, composites, ceramics, or titanium alloys.

25. The fiberoptic sensor of claim 120 wherein light provided by said light providing optical fiber is obtained from an infrared LED.

26. The fiberoptic sensor of claim 20 wherein light provided by said light providing optical fiber is obtained from a laser source.

27. The fiberoptic sensor of claim 20 wherein said plurality of light receiving optical fibers comprises at least three optical fibers.

28. A slave robot for needle insertion, the slave robot comprising:
a base component;
an MRI-compatible actuating component moving said base component;
a first sensing component sensing motion of said base components; and
a needle driving module operatively disposed on said base; and
a fiberoptic sensor operatively connected to the needle driving module; said fiberoptic sensor providing data to a robot controller; said data being utilized by said robot controller to provide force information to a master device.

29. The slave robot of claim 28 wherein said MRI-compatible actuating component is a 3 degrees of freedom (3-DOF) MRI-compatible actuating component.

30. The slave robot of claim 28 wherein said needle driving module comprises:
a stylet driving component comprising:
a stylet actuating component; and
a force sensing component; and
a cannula rotation component comprising:
a rotation actuating component; and
a rotation sensing component.

31. The slave robot of claim 28 wherein said MRI-compatible actuating components comprise piezo-electric motors.

32. The slave robot of claim 28 wherein said base and said needle driving component comprise:
a first platform;
a first linear actuating mechanism disposed on said first platform;
a first piezo-electric motor driving said first linear actuating mechanism;
a second platform disposed on said first linear actuating mechanism; said second platform being movable by said third linear actuating mechanism; and
a needle drive component mounted on said second platform; said needle drive component enabling needle insertion; the needle being operatively connected to the needle drive component.

33. The slave robot of claim 32 wherein said MRI-compatible actuating device comprises:
a vertical motion mechanism disposed on said third platform; and
a second piezo-electric motor driving said vertical motion mechanism;
said first platform being disposed on said vertical motion mechanism.

34. The slave robot of claim 33 wherein said MRI-compatible actuating device further comprises:
a fourth platform;
a second linear actuating mechanism enabling motion in one direction on said fourth platform;
a third linear actuating mechanism enabling motion in a direction perpendicular to said one direction on said fourth platform;
a second piezo-electric motor driving said second linear actuating mechanism;
a third piezo-electric motor driving said third linear actuating mechanism;
said third platform being disposed on said second and third linear actuating mechanisms;
said third platform being movable by said third and second linear actuating mechanisms.

35. The slave robot of claim 30 wherein said force sensing component comprises:
a flexure operatively coupled to said stylet driving component; said flexure configured and positioned such that axial forces induce strain in said flexure; and
a strain sensor sensing said induced strain.

36. The slave robot of claim 31 wherein said strain sensor is a fiber-optic Fabry-Perot interferometer sensor.

37. The slave robot of claim 28 wherein said fiberoptic sensor comprises:
a movable mirror mount structure;
a mirror mounted on said surface of said movable mirror mount structure;
a light providing optical fiber; said light providing optical fiber disposed along a direction of an optical axis of said mirror; said direction being determined substantially in the absence of motion of said movable mirror mount structure; one end of said light providing optical fiber providing light to said mirror;
a plurality of light receiving optical fibers; said plurality of light receiving optical fibers being disposed along a periphery of said light providing optical fiber; said plurality of light receiving optical fibers being disposed such that, when torque is transmitted to said movable mirror mount structure, a substantially asymmetric distribution of light intensity is received at said plurality of light receiving optical fibers and, when force is transmitted to said movable mirror mount structure, causing displacement along said direction of said optical axis, a substantially symmetric distribution of light intensity is received at said plurality of light receiving optical fibers.

38. A method for performing MRI-guided interventional needle procedures, the method comprising the steps of:
providing haptic feedback to and receiving position commands from an operator through a master device;
receiving, from a robot controller, position information;
providing, from the robot controller, force information to said master robot/haptic device;
receiving images from an MRI scanner;
providing, through a navigation program, trajectory planning information to said robot controller;
driving a needle utilizing a slave robot; said slave robot receiving control information from the robot controller; and
providing, from a sensor, force data to said robot controller; said data being utilized by said robot controller to provide force information to said master robot/haptic device;
thereby providing teleoperated force feedback and compensating for loss of needle tip force information.

39. The system of claim 1 where in said fiber-optic sensor comprises:
a plate disposed so instead said plate intersects the needle; said played comprising:
a flexure comprising one area of said plate;
said flexure being operatively connected to another area of said plate by a plurality of connecting members; said flexure comprising an opening; said opening being sized to receive the needle, to allow axial movement of the needle and to sense movement transverse to an axis of the needle; and
at least two strain sensors; one of said at least two strain sensors being disposed along one connecting member from said plurality of connecting members; another one of said at least two strain sensors being disposed along another connected member.

40. The system of claim 37 wherein said strain sensors comprise fiber-optic Fabry-Perot interferometer sensors.

41. The slave robot of claim 26 wherein said fiber-optic sensor comprises:
a plate disposed such that said plate intersects the needle; said plate comprising:
a flexure comprising one area of said plate;
said flexure being operatively connected to another area of said plate by a plurality of connecting members; said flexure comprising an opening; said opening being sized to receive the needle, to allow axial movement of the needle and to sense movement transverse to an axis of the needle; and
at least two strain sensors; one of said at least two strain sensors being disposed along one connecting member from said plurality of connecting members; another one of said at least two strain sensors being disposed along another connected member.

42. The system of claim 37 wherein said strain sensors comprise fiber-optic Fabry-Perot interferometer sensors.

43. The system of claim 2 wherein said slave robot operates inside an MRI scanner room.

44. The system of claim 1 wherein said master device is compatible with an MRI environment.

45. The system of claim 44 wherein said master device operates inside an MRI scanner room.

46. The system of claim 1 wherein said master device comprises:
a base component;
an MRI-compatible actuating component disposed on said based component;
one of a position and orientation sensing component operatively connected to said MRI-compatible actuating components;
a haptic interface operatively connected to said MRI-compatible actuating component; and
a force sensor operatively connected to said haptic interface.

47. The system of claim 46 wherein said force sensor is a fiber-optic force sensor.

48. A master device for MRI guided interventions, the master device comprising:
a base component;
an MRI-compatible actuating component disposed on said based component;
an actuation sensing component operatively connected to said MRI-compatible actuating components;
a haptic interface operatively connected to said MRI-compatible actuating component; and
an MRI-compatible force sensor operatively connected to said haptic interface.

49. A method for teleoperated needle insertion, the method comprising the steps of:
attaching a needle to a slave robot component;
receiving, from a remotely placed master device, displacement and force information along a needle insertion direction;
obtaining, from a robot controller, additional degree of freedom information for controlling a needle trajectory; and
providing additional degrees of freedom actuation in order to follow a predetermined needle trajectory.

50. The method of claim 49 wherein the additional degree of freedom information is determined from MRI images provided to a navigation component.

51. The method of claim 49 wherein the additional degrees of freedom include needle rotation.

52. The method of claim 51 wherein needle rotation is used to guide the trajectory based on MRI image information in conjunction with information from the master that controls needle insertion depth.
